(19) United States
(12) Patent Application Publication
(10) Pub. No.: US 2012/0315648 A1
(43) Pub. Date: Dec. 13, 2012

(54) METHOD OF IDENTIFICATION OF CELLS THAT SHOW SENSITIVITY TO MODULATION OF SIGNALING MEDIATED BY A FIBROBLAST GROWTH FACTOR RECEPTOR OR A VARIANT THEREOF

(76) Inventors: Diana Graus Porta, Basel (CH); Vito Guagnano, Basel (CH); Carlos Garcia-Echeverria, Basel (CH)

(21) Appl. No.: 13/592,709

(22) Filed: Aug. 23, 2012

Related U.S. Application Data
(63) Continuation of application No. 12/527,470, filed on Aug. 17, 2009, now abandoned.

(30) Foreign Application Priority Data
Feb. 27, 2007 (EP) 07103094.4

(51) Int. Cl. G01N 33/574; G01N 33/577

(52) U.S. Cl. 435/7.1

(57) ABSTRACT

The invention is based on the finding that cells that show (especially tyrosine) phosphorylation of FGF-R substrate 2 (FRS-2), in contrast to cells that lack such phosphorylation, allow a prediction that treatment with a modulator, especially an inhibitor, of Fibroblast Growth Factor-Receptor signaling will be successful in cells e.g. from biological samples from patients that show such phosphorylation. Therefore, the phosphorylation of FRS-2 can serve as a biomarker for the possibility of successful treatment. The invention relates to various methods, uses, kits and reagents useful in applying this biomarker.

### RELATED APPLICATIONS

This application is a continuation of U.S. application Ser. No. 12/527,470, which is a National Stage entry of International Application No. PCT/EP2008/052271, filed on Feb. 25, 2008, which claims benefit of European Application No. 07103094.4, filed on Feb. 27, 2007, which in their entirety are herein incorporated by reference.

The invention relates to a method of determining in vivo activation or inhibition of FGFR or a variant thereof and/or identification of cells, such as tumor cells (e.g. as a tumor specimen) that show sensitivity (e.g. inhibition or activation) to modulation of signaling into which a Fibroblast Growth Factor Receptor (FGF-R) or a variant thereof is involved, uses of bioreactive recognition agents for said purpose, kits comprising them, reagents for detecting them for use in identifying cells that show sensitivity to modulation, especially inhibition, of signaling into which a Fibroblast Growth Factor Receptor (FGF-R) or a variant thereof is involved, and the use of said for the manufacture of such kits, as well as other uses, methods and inventive embodiments mentioned.

Fibroblast Growth Factors (FGFs) constitute a family of over twenty structurally related polypeptides that are developmentally regulated and expressed in a wide variety of tissues or organs. FGFs stimulate proliferation, cell migration and differentiation and play a major role in skeletal and limb development, wound healing, tissue repair, hematopoiesis, angiogenesis, and tumorigenesis (reviewed in Ornitz, Novartis Found Svmp 232: 63-76; discussion 76-80, 272-82 (2001)).

The biological action of FGFs is mediated by specific cell surface receptors belonging to the receptor tyrosine kinase (RTK) family of protein kinases. These proteins consist of an extracellular ligand binding domain, a single transmembrane domain and an intracellular tyrosine kinase domain which undergoes phosphorylation upon binding of FGF. Four FGF-Rs have been identified to date: FGF-R1 (also called Flg, fms-like gene, fit-2, bFGF-R,N-bFGF-R orCek1), FGF-R2 (also called Bek-Bacterial Expressed Kinase-, KGFR, Ksam,Ksaml and Cek3), FGF-R3 (also called Cek2) and FGF-R4. All mature FGF-Rs share a common structure consisting of an amino terminal signal peptide, three extracellular immunoglobulin-like domains (Ig domain I, Ig domain II, Ig domain III), with an acidic region between Ig domains (the “acidic box” domain), a transmembrane domain, and intracellular kinase domains (Ullrich and Schlessinger, Cell 61: 203,1990; Johnson and Williams (1992) Adv. Cancer Res. 60: 1-41). The distinct FGF-R isoforms have different binding affinities for the different FGF ligands, thus FGF8 (androgen-induced growth factor) and FGF9 (glial activating factor) appear to have increased selectivity for FGF-R3 (Chellaiah et al. J Biol. Chem 1994; 269: 11620).

Recent discoveries show that a growing number of skeletal abnormalities, including achondroplasia, the most common form of human dwarfism, result from mutations in FGF-Rs. Specific point mutations in different domains of FGF-R1, FGF-R2 and FGF-R3 are associated with autosomal dominant human skeletal dysplasias classified as craniosyn-ostosis syndromes and dwarfism syndromes (Coumoul and Deng, Birth Defects Research 69: 286-304 (2003). FGF-R3 mutations-associated skeletal dysplasias include hypochondroplasia, severe achondroplasia with developmental delay and acanthosis nigricans (SADDAN) and thanatophoric dysplasia (TD) (; Webster et al., Trends Genetics 13 (5): 178-182 (1997); Tavormina et al., Am. J. Hum. Genet., 64: 722-731 (1999)). FGF-R3 mutations have also been described in two craniosynostosis phenotypes: Muenke coronal craniosynostosis (Bellus et al., Nature Genetics, 14: 174-176 (1996); Muenke et al., Am. J. Hum. Genet., 60 : 555-564 (1997)) and Crouzon syndrome with acanthosis nigricans (Meyers et al., Nature Genetics, 11 : 462-464 (1995)). Crouzon syndrome is associated with specific point mutations in FGF-R2 and both familial and sporadic forms of Pfeiffer syndrome are associated with mutations in FGF-R1 and FGF-R2 (Galvin et al., PNAS USA, 93: 7894-7899 (1996); Schell et al., Hum Mol Gen, 4: 323-328 (1995)). Mutations in FGF-Rs result in constitutive activation of the mutated receptors and increased receptor protein tyrosine kinase activity, rendering cells and tissue unable to differentiate.

Specifically, the achondroplasia mutation results in enhanced stability of the mutated receptor, dissociating receptor activation from down-regulation, leading to restrained chondrocyte maturation and bone growth inhibition (reviewed in Vajo et al., Endocrine Reviews, 21(1): 23-39 (2000)).

There is accumulating evidence for mutations activating FGF-R3 in various types of cancer.

Constitutively activated FGF-R3 in two common epithelial cancers, bladder and cervix, as well as in multiple myeloma, is the first evidence of an oncogenic role for FGF-R3 in carcinomas. In addition, a very recent study reports the presence of FGF-R3 activating mutations in a large proportion of benign skin tumors (Logie et al., Hum Mol Genet 2005). FGF-R3 currently appears to be the most frequently mutated oncogene in bladder cancer where it is mutated in almost 50% of the total bladder cancer cases and in about 70% of cases having superficial bladder tumors (Cappellen, et al., Nature Genetics 1999, 23;19-20; van Rhijn, et al., Cancer Research 2001, 61: 1265-1268; Billerey, et al, Am. J. Pathol. 2001, 158:1955-1959, WO 2004/085676). Also, overexpression of FGF-R3 has been reported in bladder cancer (superficial and invasive) (Gomez-Roman et al. Clinical Cancer Research 2005). FGF-R3 aberrant overexpression as a consequence of the t(4,14) chromosomal translocation is reported in 10-25% of multiple myeloma cases (Chesi et al., Nature Genetics 1997, 16: 260-264; Richelda et al., Blood 1997, 90 :4061-4070; Sibley et al., B J H 2002, 118: 514-520; Santra et al., Blood 2003, 101: 2374-2476). FGF-R3 activating mutations are seen in 5-10% of multiple myelomas with the t(4,14) chromosomal translocation and are associated with tumor progression (Chesi et al., Nature Genetics 1997, 16: 260-264; Chesi et al., Blood, 97 (3): 729-736 (2001); Intini, et al, BJH 2001, 114: 362-364). In this context, the consequences of FGF-R3 signaling often appear to be cell type-specific. In chondrocytes, FGF-R3 hyper-activation results in growth inhibition (reviewed in Omitz, 2001), whereas in the myeloma cell it contributes to tumor progression (Chesi et al.,2001).

The inhibition of FGF-R3 activity has been found to represent a means for treating T cell mediated inflammatory or autoimmune diseases, as for example in treatment of T-cell mediated inflammatory or autoimmune diseases including but not limited to rheumatoid arthritis (RA), collagen II arthritis, multiple sclerosis (MS), systemic lupus erythematosus (SLE), psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis. See WO 2004/110487. Disorders resulting from FGF-R3 mutations are described also in WO 03/023004 and WO 02/102972.

Among the diseases promoted by FGF-R3 and also other FGF-Rs (especially in connection with e.g. aberrant FGF23 serum levels), further Autosomal Dominant Hypophosphatemic Rickets (ADHR), X-chromosome linked hypophosphatemic rickets (XLH), tumor-induced Osteomalacia (TIO), fibrous dysplasia of the bone (FH) are to be mentioned (see also X. Yu et al., Cytokine & Growth Factor Reviews 16 , 221-232 (2005), and X. Yu et al., Therapeutic Apheresis and Dialysis 9(4), 308-312 (2005)).

Gene amplification and /or overexpression of FGF-R1, FGF-R2 and FGF-R4 have been implicated in breast cancer (Penault-Llorca et al., Int J Cancer 1995; Theillet et al., Genes Chrom. Cancer 1993; Adnane et al., Oncogene 1991; Jaakola et al., Int J Cancer 1993; Yamada et al, Neuro Res 2002). Overexpression of FGF-R1 and FGF-R4 is also associated with pancreatic adenocarcinomas and astrocytomas (Kobrin et al., Cancer Research 1993; Yamanaka et al., Cancer Research 1993; Shah et al., Oncogene 2002; Yamaguchi et al., PNAS 1994; Yamada et al., Neuro Res 2002). Prostate cancer has also been related to FGF-R1 overexpression (Giri et al., Clin Cancer Res 1999).

FGFs/FGF-Rs are also involved in angiogenesis. Therefore, targeting the FGF-R system is also foreseen as an anti-angiogenic therapy to treat primary tumors, as well as metastasis. (see e.g. Presta et al., Cytokine & Growth Factors Reviews 16, 159-178 (2005)).

Mutations, especially in FGF-R3 (e.g. FGF-R3b) have also been described to be responsible for constitutive activation of these receptors in the case of oral squameous cell carcinoma (see e.g. Y. Zhang et al, Int. J. Cancer 117, 166-168 (2005).

Enhanced (especially bronchial) expression of FGF-Rs, especially FGF-R1, has been reported to be associated with Chronic Obstructive Pulmonary Disease (COPD) (see e.g. A. Kranenburg et al., J. Pathol. 206, 28-38 (2005)).

Methods of antagonizing FGF-Rs, especially FGF-R1 or FGF-R4, have also been described to be useful in the treatment of obesity, diabetes and/or diseases related thereto, such as metabolic syndrome, cardiovascular diseases, hypertension, aberrant cholesterol and triglyceride levels, dermatological disorders(e.g. infections, varicose veins, Acanthosis nigricans, eczema, exercise intolerance, diabetes type 2, insulin resistance, hypercholesterolemia, cholelithiasis, orthopedic injury, thromboembolic disease, coronary or vascular restriction (e.g. atherosclerosis), daytime sleepiness, sleep apnoea, end stage renal disease, gallbladder disease, gout, heat disorders, impaired immune response, impaired respiratory function, infections following wounds, infertility, liver disease, lower back pain, obstetric and gynecological complications, pancreatitis, stroke, surgical complications, urinary stress incontinence and/or gastrointestinal disorders (see e.g. WO 2005/037235 A2).

Acidic Fibroblast Growth Factor (especially FGF-1) and FGF-R1 has also been described to be involved in aberrant signaling in retinoblastoma, leading to proliferation upon binding of FGF-1 (see e.g. S. Siffroi-Fernandez et al., Arch. Ophthalmology 123, 368-376 (2005)). The growth of synovial sarcomas has been shown to be inhibited by disruption of the Fibroblast Growth Factor Signaling Pathway (see e.g. T. Ishibe et al., Clin. Cancer Res. 11(7), 2702-2712 (2005)).

Further, FGF-R involvement in the case of thyroid carcinoma could be demonstrated.

In all the cases mentioned above, the modulation of an aberrant activity of FGF-R signaling (especially the inhibition of an activity of such a kinase) can be expected reasonably to be useful in the treatment of the diseases mentioned.

However, it would be desirable to have an indication when treatment with drugs that modulate, e.g. inhibit or activate, FGF-R signaling can be expected to be useful and further to have a marker allowing to monitor FGF-R modulation and whether treatment with such modulating compounds is effective or not.

Having an indication of when treatment with drugs inhibits FGF-R signaling is especially desirable.

FRS-2, also called SNT1, is a lipid-anchored adaptor protein that serves as the primary link between FGF-R activation and intracellular signaling pathways (Lin et al., Mol. Cell Biol. 18: 3762-3770, 1998; Xu et al., J. Biol. Chem. 273: 17987-17990, 1998 ; Dhalluin et al., Mol. Cell 6 : 921-929, 2000 ; Ong et al., Mol. Cell Biol. 20: 979-89, 2000). FRS-2 comprises a receptor recognition sequence of the phosphotyrosine binding class (PTB) which constitutively associates with the juxtamembrane region of the FGF-Rs, and an effector domain with multiple tyrosine and serine phosphorylation sites. FGF-R activation leads to phosphorylation of FRS-2 tyrosine residues, to which Grb2 and the tyrosine phosphatase Shp2 are subsequently recruited initiating MAPK and PI3K signaling (Xu et al. 1998, loc. cit.; Ong et al. 2000, loc. cit.; Hadari et al., Proc. Natl. Acad. Sci. USA 98: 8578-83, 2001). The importance of FRS-2 in

FGF signaling is reflected in the embryonic lethal phenotype observed during mouse development after disruption of the FRS-2 gene. In addition, FRS-2-deficient mouse embryo fibroblasts show an impairment of FGF-induced migration, proliferation and MAK activation (Hadari et al. 2001).

### GENERAL DESCRIPTION OF THE INVENTION

Surprisingly, it has now been found that the (especially tyrosine) phosphorylation status of FRS-2 can serve as a biomarker for the efficiency of such modulating compounds against the mentioned (especially proliferative) diseases and disorders: It is especially shown that FRS-2 is highly tyrosine phosphorylated in such cells that are sensitive to inhibition of FGF-R signaling but not in those lines that are independent of FGF-Rs for proliferation

This invention is thus based on the finding that FGFR inhibition by FGFR modulators results in a reduction in the level of phosphorylated FRS-2. In proliferating (e.g. tumor) cells the degree of tyrosine phosphorylation of FRS-2 in cells that respond to inhibition of FGF-R signaling is diminished in the presence of FGF-R signalling inhibitors, while in cells that do not respond to inhibition the level of tyrosine phosphorylation is not detectable, that is very low to zero - or more generally not susceptible to changes by addition of a modulator, especially an inhibitor, and that only cells that show tyrosine phosphorylation of FRS-2 can be expected to show sensitivity to inhibition of FGF-R signaling.

Alternatively, where activation of the FGF-R signalling is desired (e.g. in the case of wound healing), it is also possible to examine whether activators (such as FGF derivatives or the like) lead to an increase of the phosphorylation of FRS-2, a variant thereof or a phosphortyrosine comprising fragment thereof, thus indicating a (then desirable) activation of FGF-R signalling.

Therefore, the tyrosine phosphorylation degree (especially the presence of tyrosine phosphorylation) of FRS-2 in proliferating cells can be used to distinguish cells that are able to react on the treatment with FGF-R signalling modulators, especially inhibitors, from cells that would be non-responsive to such treatment. Thus, the tyrosine phosphorylation status of FRS-2 in cells is a biomarker for the possibility to modulate, especially inhibit (e.g. undesired) cell proliferation by inhibitors of FGF-R signalling. The levels of FRS-2 tyrosine phosphorylation may also be used to determine ex-vivo the activity of modulators of FGFRs and variants thereof.

Further, the determination of FRS-2 tyrosine phosphorylation (or synonymously of phosphorylated forms of FRS-2, a variant thereof or a tyrosine comprising fragment thereof) is inter alia useful in the identification of compounds/drugs that modulate FGF-R activity (especially regarding the activity of and may also be applied in a diagnostic method for identifying patients that may benefit from treatment with FGF-R modulators, especially inhibitors, as well as for the monitoring of the efficiency of the treatment.

### DETAILED DESCRIPTION OF THE INVENTION

In a first embodiment, the invention relates to a method of identification of (preferably isolated, e.g. in cell culture or cell suspensions) cells (including isolated cells, or cells from isolated tissues and/or organs, generally from biological samples, most preferably from patients) that show sensitivity to modulation, especially inhibition, of signaling into which a Fibroblast Growth Factor Receptor (FGF-R) or a variant thereof is involved, comprising determining the tyrosine phosphorylation status of an FGF-R substrate 2 (FRS-2), a variant thereof or a tyrosine comprising fragment thereof in a biological sample as biomarker for such sensitivity to inhibition.

In a further embodiment, the invention relates to a method of using or to the use of phosphorylation (especially phosphotyrosine) identification in FRS-2, a variant thereof of a tyrosine comprising fragment thereof, as a biomarker for cells or tissues or organs, especially from a biological sample from a patient, that show hyperactive, especially constitutively activated, FGF-R signaling, especially that are treatable with inhibitors of FGF-R or a variant thereof and that are responsive to such inhibitors, said method or use comprising determining the presence of phosphotyrosine in FRS-2, in a variant thereof or in a tyrosine comprising fragment thereof from a biological sample with a biospecific recognition reagent capable of recognizing phosphotyrosine in FRS-2, a positive finding of phosphorylation in FRS-2 indicating hyperactive, especially constitutively activated, FGF-R signalling, and preferably, in order to distinguish cells or tissues or organs that are responsive from such cells or tissues or organs that are non-responsive to inhibitors of signaling into which a Fibroblast Growth Factor Receptor (FGF-R) or a variant thereof is involved, comparing the phosphorylation status in the absence and in the presence of an inhibitor of signaling mediated by FGF-R or a variant thereof, a decrease in the phosphorylation in the presence of an inhibitor indicating such responsiveness.

The invention also relates to a method for the identification of (preferably isolated, e.g. in cell culture or cell suspensions) cells (including isolated cells, or cells from isolated tissues and/or organs, generally from a biological sample, especially from a patient) that show sensitivity to modulation, especially inhibition, of signaling into which a Fibroblast Growth Factor Receptor (FGF-R) or a variant thereof is involved, comprising
a) contacting the biological sample with a biospecific recognition reagent capable of recognizing FRS-2 or a variant or a tyrosine comprising fragment thereof and
b) determining the phosphorylation status of the tyrosine in said FRS-2, a variant thereof or a tyrosine comprising fragment thereof with a biospecific recognition reagent capable of recognizing said phosphorylation status, and
c) correlating the phosphorylation status to the sensitivity to inhibition of signaling into which a Fibroblast Growth Factor Receptor (FGF-R) or a variant thereof (especially a hyperactive, e.g. constitutively active form) is involved and/or the condition status and/or treatment efficacy.

In a further embodiment, the invention also relates to a kit for use in the identification of a biological sample, especially from a patient, which is sensitive to modulation, especially inhibition, of signaling into which an FGF-R or a variant thereof is involved, especially for use in the identification of patients where treatment with an FGF-R inhibitor is useful, comprising means for determining the phosphorylation status of an FRS-2, a variant thereof or a tyrosine comprising fragment thereof, especially means for identifying phosphorylated, more especially tyrosine phosphorylated FRS-2, a variant thereof or a tyrosine comprising fragment thereof, in a biological sample, preferably from a patient, as biomarker for such sensitivity to modulation, especially inhibition; where preferably if the kit allows a positive finding of phosphorylation in the absence of modulation, more preferably in the absence of FGF stimulation, that is yet more preferably in the case of constitutive activation of FGF-R signaling, this indicates sensitivity to inhibition, especially if the phosphorylation is decreased in a sample treated with an inhibitor as compared to a sample without inhibitor treatment.

Yet a further embodiment of the invention relates to the use of a biospecific recognition reagent (especially an antiphosphotyrosine antibody) capable of recognizing a phosphorylated form of FRS-2 or of a variant or of a tyrosine comprising fragment thereof (especially of recognizing phosphotyrosine therein) or to said biospecific recognition reagent as such for use in the identification of cells, especially from a biological sample, especially from a patient, that show sensitivity to modulation, especially inhibition, of signaling into which a Fibroblast Growth Factor Receptor (FGF-R) or a variant thereof is involved (more especially for use in the identification of patients where treatment with an FGF-R inhibitor is useful), where said use preferably comprises determining the phosphorylation status of said FRS-2 or variant or fragment thereof; where a finding of phosphorylation in the absence of modulation preferably means that sensitivity to said inhibition can be expected; preferably for use in the identifycation of a hyperactivity, especially an FGF-independent activation, more especially a constitutive activation of FGF-R signaling. Preferred is the use or the biospecific recognition reagent for use in the identification of a condition in a patient that is responsive to the treatment with an inhibitor of FGF-R signaling, comprising identification of phosphorylation, especially tyrosine phosphorylation, in FRS-2, a variant thereof or a phosphotyrosine comprising fragment thereof in a biological sample from said patient.

Another embodiment of the invention relates to a method for the determination of FRS-2 phosphorylation (or synonymously of phosphorylated forms of FRS-2, a variant thereof or a tyrosine comprising fragment thereof) for use in the identification of a modulator (a compound or drug that modulates the activity) of signalling into which a Fibroblast Growth Factor Receptor (FGF-R) or a variant thereof is involved, comprising determining the FRS-2 phosphorylation status in the absence and presence of such a modulator and, if a decrease of said phosphorylation is found in the case of addition of said (possible) modulator, assigning the modulator to the class of inhibitors, if an increase of said phosphorylation is found in the case of addition of the modulator, assigning the modulator to the class of activators of the signalling, respecttively; especially for the identification of cells that show a hyperactivity, especially an FGF-independent activation, more especially a constitutive activation of FGF-R signaling.

Another embodiment of the invention relates to a diagnostic method or use of a biospecific recognition reagent (especially an antiphosphotyrosine antibody) capable of recognizing a phosphorylated form of FRS-2, of a variant thereof or of a tyrosine comprising fragment thereof, or said biospecific recognition reagent for use, in identifying a patient that may benefit from treatment with FGF-R modulators, especially inhibitors, especially comprising determining whether such a phosphorylation is present without inhibitor (especially a positive finding of such phosphorylation indicating that a patient may benefit from treatment with an inhibitor) and preferably whether it is decreased in the presence of the inhibitor in a biological sample from such a patient, or in the monitoring of the efficiency of a treatment with such FGF-R modulators, especially inhibitors, treatment, comprising determining whether such a phosphorylation if positively found without treatment is changed, especially decreased in the presence of the modulator, especially inhibitor, in a biological sample from such a patient; preferably for use in the identification of a hyperactivity, especially an FGF-independent activation, more especially a constitutive activation of FGF-R signaling in a biological sample of such a patient.

Still another embodiment of the invention relates to a method of diagnosing an (especially proliferative) disease (or a patient) responsive to treatment with an inhibitor of FGF-R signaling, comprising identifying a (preferably tyrosine) phosphorylated form of FRS-2, of a variant thereof or of a tyrosine comprising fragment thereof in a biological sample from a patient, preferably with a biospecific recognition reagent capable of recognizing a phosphorylated form of FRS-2, of a variant thereof or of a tyrosine comprising fragment thereof, or to said biospecific recognition reagent for use in said method of diagnosing; where preferably the use in the identification of a hyperactivity, especially an FGF-independent activation, more especially a constitutive activation of FGF-R signaling.

Still another embodiment of the invention relates to a method of diagnosing a proliferative disease not susceptible to treatment with an inhibitor of FGF-R signaling, comprising identifying the absence of a phosphorylated form of FRS-2, of a variant thereof or of a tyrosine comprising fragment thereof in a biological sample, preferably with a biospecific recognition reagent capable of recognizing a phosphorylated form of FRS-2, of a variant thereof or of a tyrosine comprising fragment thereof, or to said biospecific recognition reagent for use in said method of diagnosing.

Another embodiment of the invention relates to a method of monitoring the response to a therapy for treating a disorder dependent on FGF-R signaling in a patient, comprising obtaining a biological sample from said patient before said therapy, determining the presence of a phosphorylated form of FRS-2, of a variant thereof or of a tyrosine comprising fragment thereof, especially the degree of phosphorylation, respectively, and obtaining one or more further biological samples after the start of said therapy and determining whether the degree of phosphorylation of said FRS-2, of said variant thereof or of said tyrosine comprising fragment has changed, especially been decreased, where a decrease in the degree of phosphorylation indicated a successful treatment.

Still a further embodiment of the invention relates to the use of a biospecific recognition reagent capable of recognizing phosphorylated FRS-2 or a variant or a tyrosine comprising fragment thereof for the manufacture of a diagnostic for the identification of cells from cells or tissues from a biological sample that are sensitive to modulation of signaling into which a Fibroblast Growth Factor Receptor (FGF-R) or a variant thereof is involved, said identification comprising determining the phosphorylation status of an FGF-R substrate 2 (FRS-2), a variant thereof or a tyrosine comprising fragment thereof.

In yet a further embodiment, the invention relates to the use of a biospecific recognition reagent capable of recognizing phosphorylated FRS-2, a variant thereof or a fragment thereof to identify cells useful for the identification of compounds that modulate FGF-R signaling.

A further embodiment of the invention relates to a method for identifying cells that proliferate requiring, especially constitutive, FGF receptor activation for proliferation and are responsive to inhibition of FGF-R signaling, comprising
a) subjecting a sample of isolated cells or tissue to a medium in the absence of an FGF-R inhibitor and a parallel sample in the presence of an FGF-R receptor inhibitor in the absence of FGF,
b) at least partially purifying FRS-2, a variant thereof or a tyrosine comprising fragment thereof from said samples;
c) determining the phosphorylation status of FRS-2 in said samples; and
d) comparing the phosphorylation status in the samples treated with that in the samples not treated with the inhibitor, a decrease of phosphorylation in the presence of an inhibitor indicating cells that are appropriate for identifying inhibitors useful in the treatment of a condition that includes hyperactivity of FGF-R signaling.

The method is useful for identifying cells that proliferate by FGF dependent or FGF independent, especially constitutive, FGF receptor activation for proliferation.

Yet a further embodiment of the invention relates to a method of using or the use a biospecific recognition reagent capable of recognizing phosphorylated FRS-2, a variant thereof or a tyrosine comprising fragment thereof, for the identification of potential inhibitors of FGF-R dependent signaling, comprising determining with said reagent the phosphorrylation status of FRS-2, a variant thereof or a tyrosine comprising fragment thereof, from a biological sample and, in the case of finding of phosphorylation, comparing the degree of phosphorylation in the presence of a test compound with that in its absence, a decrease in the phosphorylation indicating the usefulness of the test compound as inhibitor of FGF-R dependent signaling.

All the preceding methods, uses, reagents, kits and other embodiments of the invention preferably allow to identify patients with a condition, especially disorder or disease, that can be expected to be responsive to treatment with modulators of FGF-R signaling, especially in case of a positive identification of phospho-tyrosine in FRS-2, a variant thereof of a fragment thereof comprising tyrosine.

The general terms used hereinbefore and hereinafter preferably have within the context of this disclosure the following meaning, unless otherwise indicated—any one or more of the more general expression used herein, especially in the claims, can, independently of other terms, be replaced with a more specific definition provided below, thus defining a preferred embodiment of the invention:

Cells that show “sensitivity to modulation of signaling into which a Fibroblast Growth Factor Receptor (FGF-R) or a variant thereof is involved” preferably means cells that are responsive to treatment with a modulator, especially inhibitor, of said signaling, and that are comprised in a biological sample from a patient. These cells show a change in phosphorylation of FRS-2, a variant thereof or a tyrosine comprising fragment thereof in the presence of a modulator of FGF-R when compared with the phosphorylation in the absence of a modulator—especially in the case where the modulator is an inhibitor an increase in phosphorylation, or more preferably where the modulator is an inhibitor a decrease in phosphorylation is found. The modulator, preferably inhibitor, is preferably a molecule binding to FGF-R or a variant thereof and preferably inhibiting its (preferably tyrosine) protein kinase activity regarding FRS-2 or a variant thereof.

“Phosphorylation status” (or “phosphorylation degree”) refers to the absence or partial or complete presence of phosphorrylated serine, threonine and/or (preferably and only) tyrosine molecules in the primary amino acid sequence of FRS-2, a variant thereof or a tyrosine comprising fragment thereof. The phosphorylation status is, according to the invention, a means to distinguish biological samples e.g. from patients which suffer from a condition, e.g. a disease or disorder, that is dependent on (e.g. constitutive) FGF-R signaling (where phosphorylation can be shown to be present) from samples where no such dependency is given (where no or only weak phosphorylation can be shown to be present).

Especially by comparing the phosphorylation status of biological samples after incubation in the presence and in the absence of a modulator (especially inhibitor) of FGF-R signaling, it is possible to distinguish biological samples in which the FGF-R signaling can be modulated (especially inhibited) (and which are thus responsive to treatment with such modulators, especially inhibitors, which in the case of inhibitors is the case if there without inhibitor phosphorylation is found and this phosphorylation is diminished or removed in the presence of an inhibitor) from biological samples where such modulation, especially inhibition, does not affect phosphorylation status (that is, in the case of inhibitors, where no phosphorylation is present both in the presence or absence of an inhibitor or where no change of a given phosphorrylation is seen comparing the samples with or without inhibitor).

Most preferably, the invention allows to identify cells which, due especially to hyperactivity and most especially constitutive activation of FGF-R signaling, proliferate unduly and therefore show tyrosine phosphorylation in FRS-2 or a variant thereof and thus are (as can be distinguished further by examining their phosphorylation status both in presence and absence of an inhibitor of FGF-R signaling) expected to be responsive to treatment with an inhibitor of FGF-R signaling, allowing to distinguish them from cells where such phosphorylation is absent and in which therefore a treatment with inhibitors of FGF-R signaling is not expected to be successful.

Generally, thus a finding of phosphorylation (especially tyrosine phosphorylation) of FRS-2 or variants or tyrosine comprising fragments thereof which is also present in the absence of FGF stimulation or a biological sample thus is a highly preferred biomarker according to the invention, and all inventive embodiments very most especially refers to the finding of such cells that show this type of hyperactivity, especially constitutive activity, of FGF-R or variants thereof.

If the biological samples are from a patient, the phosphorylation status (which term can also be replaced with the term “phosphorylation degree” herein) and the presence of phosphorylation or lack of variation in the presence and absence of a modulator (especially an inhibitor) thus allows to decide whether the patient can profit from treatment with a modulator, especially inhibitor, or not (those where a diminished phosphorylation or no phosphorylation is found in the presence of inhibitor while a higher phosphorylation is found in the absence of inhibitor can be predicted to profit from treatment with such an inhibitor).

“Phosphorylation”, wherever used herein, is preferably referring to tyrosine phosphorylation.

Patients are preferably warm-blooded animals, more preferably mammalians, most preferably humans, prone to be suffering or suffering from a condition or disorder that (at least partially) depends on the over-activity of FGF-R signaling, especially due to hyperactivity, especially due to constitutive activation, or for other reasons (e.g. enlarged susceptibility to FGFs and/or increased FGF levels, increased FGF-R biosynthesis or the like).

Among the conditions, especially diseases or disorders selected from the following are important:

Skeletal abnormalities, including achondroplasia, the most common form of human dwarfism, skeletal dysplasias classified as craniosynostosis syndromes and dwarfism syndromes, e.g. hypochondroplasia, severe achondroplasia with developmental delay and acanthosis nigricans (SADDAN) and thanatophoric dysplasia, Muenke coronal craniosynostosis, Crouzon syndrome with acanthosis nigricans, Autosomal Dominant Hypophosphatemic Rickets, X-chromosome linked hypophosphatemic rickets (XLH), tumor-induced Osteomalacia, fibrous dysplasia of the bone.

Autoimmune diseases, e.g. rheumatoid arthritis, collagen II arthritis, multiple sclerosis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis, oral squameous cell carcinoma.

Chronic Obstructive Pulmonary Disease, obesity, diabetes and/or diseases related thereto, such as metabolic syndrome, cardiovascular diseases, hypertension, aberrant cholesterol and triglyceride levels, dermatological disorders(e.g. infections, varicose veins, Acanthosis nigricans, eczema, exercise intolerance, diabetes type 2, insulin resistance, hypercholesterolemia, cholelithiasis, orthopedic injury, thromboembolic disease, coronary or vascular restriction (e.g. atherosclerosis), daytime sleepiness, sleep apnoea, end stage renal disease, gallbladder disease, gout, heat disorders, impaired immune response, impaired respiratory function, infections following wounds, infertility, liver disease, lower back pain, obstetric and gynecological complications, pancreatitis, stroke, surgical complications, urinary stress incontinence and/or gastrointestinal disorders.

Especially important are proliferative disorders, especially cancer or tumor diseases of tissues, organs or blood cells, such as epithelial cancer of the bladder or the cervix, multiple myeloma, skin tumors, breast cancer, pancreatic adenocarcinoma, astrocytoma, prostate cancer, solid tumors where angiogenesis play a role, retinoblastoma, synovial sarcoma, thyroid carcinoma, further melanoma, malignant lymphoma, gastrointestinal cancer, other pancreatic cancer, lung cancer, esophagus cancer, liver cancer, ovarian cancer, uterine cancer, prostate cancer, brain tumor, Kaposi's sarcoma, angioma, osteosarcoma, muscle sarcoma, glioblastoma, 8p11 myeloproliferative disorder or leukemias; or the like.

FGF-Rs are high affinity receptors for the FGFs (which also show variants, e.g. more than 20 genes and several isoforms) which can be found in various variants.

The term “FGF-R”, especially FGF-R1, FGF-R2, FGF-R3 and FGF-R4, as used herein includes all these variants, especially those that still, constitutionally active or active due to binding (preferably with a dissociation constant of 10⁻³ or stronger, more preferably of 10⁻⁵ or stronger, yet more preferably of 10⁻⁷ or stronger) of one or more of the 22 Fibroblast Growth Factors (FGF) known, are able to phosphorylate FRS2 to yield the phosphotyrosine form thereof, as demonstrable with a antiphosphotyrosine antibody (such as 4G10) and especially by the assays in the examples, or that show activity (tyrosine phosphorylation) in assays corresponding to those mentioned in WO 2006/000420 (which is therefore included by reference regarding these assay) as FGF-R3 (Cellular Assay) or FGF-R3 (Enzymatic Assay).

Preferably, the variants comprise (preferably consist of) a sequence that is (on amino acid basis) about (meaning where used especially ±10 percent of the respective numerical value to which “about” is attached, or preferably exactly the value) 70% or more identical, more preferably at least about 85% or more identical, yet more preferably about 90% or more identical, still more preferred about 95% or more identical, very preferred 98% or more identical. The percentage of sequence identity, also termed homology, between FGF-R, especially FGF-R1, FGF-R2, FGF-R3 or FGF-R4 and a variant thereof is preferably determined by a computer program commonly employed for this purpose, such as the Gap program (Wisconsin Sequence Analysis Package, Version 8 for Unix, Genetics Cmputer Group, University Research Park, Madison Wis., USA, which uses the algorithm of Smith and Waterman (Adv. Appl. Math. 2: 482-489 (1981)., especially using an affine gap search with a gap open penalty of 12 and a gap extension penalty of 1.

A preferred basis, besides the original sequences of FGF-R1, FGF-R2, FGF-R3 and FGF-R4, for comparison regarding identity of all the sequence variants given above are for—FGF-R1 the protein sequence given in

http://www.ncbi.nlm.nih.gov/entrez/viewer.fcgi?val=M34185; (Accession No. M34185);
for FGF-R2 the protein sequence given in
http://www.ncbi.nlm.nih.gov/entrez/viewer.fcgi?db=nucleotide&val=108773805 (Accession No. NM_{—}022970 NM_{—}022969);
for FGF-R3, the protein sequence given in
http://www.ncbi.nlm.nih.gov/entrez/viewer.fcgi?db=nucleotide&val=13112047 (Accession No. NM_{—}022965);
and for FGF-R4 the protein sequence given in
http://www.ncbi.nlm.nih.gov/entrez/viewer.fcgi?db=nuccore&val=47524176 (Accession No. NM_{—}022963).

The term “variants” includes various forms, such as especially isoforms, mutants (non-conservative or especially conservative, e.g. substitution, deletion and/or addition of one or more, e.g. up to ten, amino acids, including also inversions), allelic variants, polymorphism based variants, fusion proteins, truncated forms (e.g. lacking 10 to 50 amino acids in comparison to the full sequence) or the like. Especially preferred are the mutants, isoforms, translocation variants and the like mentioned specifically in the next paragraph:

There are four general genes for FGF-R, namely for FGF-R1, FGF-R2, FGF-R3 and FGF-R4, each including several isoforms (e.g. FGF-R1: a, b, c; FGF-R2: b,c; FGF-R3: b,c; FGF-R4: truncated form). Polymorphism is given, mutants exist (e.g. for FGF-R3: R248C (#), S249C (#), P250R, N328I, G370/372C (#), S371/373C, Y373/375C (#), G375/377C, G380/382R (#), A391/393E (#), 1538/540V*, N540/542K,T,S or V, K650/652E (#), K650/652M (#), K650/652Q (#), K650/652N, K650/652T (#), X807/809C,G,L,R,W or the like, many of which are involved in disease such as cancer formation, for example those marked with # can be found e.g. in bladder cancer, and/or in skeletal dysplasias, comparable mutations are also present for FGF-R1 and FGF-R2; fusion proteins exist as a consequence of translocations, e.g. FGF-R1: Bcr-FGF-R1, ZNF198-FGF-R1, CEP110-FGF-R1, FOP-FGF-R1, Trim-FGF-R1, MYO18A-FGF-R1, TIF1-FGF-R1; FGF-R2: Tel-FGF-R3 (see e.g. Eswarakumat et al., J. Cytokine & Growth Factor Reviews 16 (2005), 139-149).

Thus, generally also mutants (including substitutions, deletions, insertions), isoforms, polymorphism variants and fusion proteins are encompassed, e.g. in one or more of the following parts of the FGF-R proteins: Ig domain I, acidic box, Ig domain II, Ig domain III, Transmembrane domain, Kinase 1, Kinase insert and/or Kinase 2.

Also different FRS-2 types, also named SNTs (Suc-associated neurotrophic factor-induced tyrosine-phosphorylated targets), are found. The FRS-2 family of proteins consists basically of FRS-2α (SNT1) and FRS-2β (SNT2) which are lipid-anchored multisubstrate adaptor molecules that recruit the SH2 domain-containing protein Grb1 and the SH2-containing protein tyrosine phosphatase (PTP) SHP-2. Tyrosyl phosphorylation of FRS-2α is critical for the initiation of FGF-R signaling. Where an FRS-2 is mentioned in the present disclosure, this also relates to variants of FRS-2-α (SNT1) and FRS-2β (SNT2) which still are able to bind to FGF-R1, FGF-R2, FGF-R3 and/or FGF-R4, that is, especially FRS-2 variants of that kind that are 70% or more identical, more preferably at least about 85% or more identical, yet more preferably about 90% or more identical, still more preferred about 95% or more identical, very preferred 98% or more identical, to FRS-2α or FRS-2β. The percentage of sequence identity, also termed homology, between between FRS-2α or FRS-2β and a variant thereof is preferably determined by a computer program commonly employed for this purpose, such as the Gap program (Wisconsin Sequence Analysis Package, Version 8 for Unix, Genetics Cmputer Group, University Research Park, Madison Wis., USA, which, uses the algorithm of Smith and Waterman (Adv. Appl. Math. 2: 482-489 (1981), especially using an affine gap search with a gap open penalty of 12 and a gap extension penalty of 1.

Thus, variants of FRS-2 (FRS-2α or FRS-2β) include especially isoforms, mutants (non-conservative or especially conservative, e.g. substitution, deletion and/or addition of one or more, e.g. up to ten, amino acids, including also inversions), allelic variants, polymorphism based variants, fusion proteins, truncated forms (e.g. lacking 10 to 50 amino acids in comparison to the full sequence) or the like. Especially preferred are FRS-2α and FRS-2β; any variants must include at least one site accessible to phosphorylation, especially a tyrosine.

Tyrosine comprising fragments of FRS-2 or a variant thereof are such peptides that have (e.g. by proteolytic site specific cleavage with proteases, such as Submaxillarus protease, Staphylococcus aureus V8 protease, Pepsin, Asp-N-protease, chymotrypsin or trypsin or by chemical cleavage e.g. with bromocyan, preferably each obtained by proteolytic or chemical cleavage of FRS-2 or a variant thereof obtained from a biological sample, especially cells or tissues or organs, very especially a tumor) one or more tyrosine moieties that can be phosphorylated in their chain and can still be recognized by biospecific recognition reagents that are capable to distinguish unphosphorylated and phosphorylated forms of these peptides, especially also from other peptides e.g. from other proteins. Preferably, the fragments have 5 or more, more preferably 10 or more contiguous amino acids, yet more preferably 20 or more, especially 50 or more, of the original sequence of the FRS-2 cleaved.

A “phosphorylated form” of FRS-2, of a variant thereof or of a tyrosine comprising fragment thereof is preferably one that is phosphorylated at one or more serine, threonine or (especially) tyrosine moieties in the primary amino acid structure of said FRS-2, variant or fragment.

The term “biological samples” can, for example, refer to body liquids, such as sputum, blood, blood plasma, blood serum, synovial fluid, intraperitoneal fluid, intrapulmonal fluid or urine, or more preferably cells, cell components or tissue or organ specimens (including samples from organs or especially tumors), or mixtures of two or more thereof, such as tissue or organ samples or cells or cell lysates from cells, organs or tissues to be examined (e.g. from cells, tumors or other tissues or organs affected or presumed to be affected). Preferably, isolated biological samples are meant. These may derive from cultures or may preferably have been obtained from patients. In the methods and uses according to the invention, usually isolated biological samples are used, that is the methods and uses preferably take place in the absence of a patient, e.g. in a separate laboratory or the like. Thus the steps of obtaining a biological sample and of its examination can be and preferably are separate, in this case, and the methods or uses according to the invention are independent of the type of sampling or sample. Where the term “cells” is used herein, this refers to cells, cell fragments or cell lysates from biological samples as just defined.

The methods or uses according to the invention, in one preferred embodiment, do not include the step of obtaining the sample from a patient, that is the purely in vitro method or use, that is one outside and in the absence of the body of a patient. On the other hand, also those embodiments where this obtaining of a sample is also encompassed are a preferred embodiment of the invention where allowable.

A “biospecific recognition reagent” can be an antibody, in specific cases (especially as secondary or tertiary labeled biospecific recognition molecules) it may also be an antibody binding protein (e.g. protein A or protein G from bacteria), or it can be aptamers (that include double-stranded DNA or single-stranded RNA molecules that bind to specific molecular targets) or affibodies (protein binding polypeptides that can be selected to the desired protein and can, for example, be isolated from combinatorial protein libraries).

The general term “antibody”, within the present disclosure and if not specified otherwise, is intended to include polyclonal or monoclonal antibodies, bispecific antibodies, humanized antibodies, chimeric antibodies, single chain antibodies, or fragments of any one or more of these forms that still recognize, especially show a (substantially or fully selective) binding affinity to (preferably with a dissociation constant K of 10⁻⁴ or lower, more preferably of 10⁻⁶ or lower, still more preferably of 10⁻⁸ or lower), FRS-2 and/or variants and/or tyrosine comprising fragments thereof, including one or both of conformational or (preferably) primary structure related (e.g. phosphotyrosine comprising) epitopes. Thus, “antibody” refers especially to a protein functionally defined as a binding protein (a molecule able to bind to a specific (conformational and/or primary structure related) epitope on an antigen) and structurally defined as comprising an amino acid sequence that is recognized by a person skilled in the art as being derived from the framework region of an immunoglobulin encoding gene. Structurally, the simplest naturally occurring antibody (e.g. IgG) comprised four polypeptide chains, two copies of heavy (H) chain and two copies of light (L) chain, all covalently linked by disulfide bonds. Specificity of binding to the epitope is found in the variable (V) region of the H and L chains. Regions of the antibodies that are primarily structural are constant (C). The term “antibody” includes whole antibodies, still binding fragments, modifications or derivatives of an antibody. It can also be a recombinant product, or a bispecific antibody or chimeric antibody, such as a humanized antibody. Antibodies can be a polyclonal mixture or (more than one or especially one) monoclonal. They can be intact immunoglobulins derived from a natural source or natural sources and can be immunoreactive (binding) portions of intact immunoglobulins. Antibodies may show a variety of forms (derivatives), including, for example, Fv (consisting of V_{L} and V_{H} domains), a dAB fragment (consisting of a V_{H} domain; see Ward et al., Nature 341: 544-546, 1989), an isolated complementarity determining region (CDR), Fab (consisting of the V_{L}, V_{H}, C_{L} and C_{H1} domains), and F(ab)₂ (a bivalent fragment comprising two Fab fragments linked by a disulfide bridge at the hinge region) as well as in single chains. Single chain antibodies (SCA), in which genes for a heavy chain and a light chain are combined into a single coding sequence, may also be used. Some SCA are recombinant molecules containing the variable region of the light chain, the variable region of the heavy chain and a suitable polypeptide linker linking them. Recognizing or recognition especially means that there is a (preferably specific, e.g. 100-fold, preferably 1000-fold, more preferably 10,000-fold or in each case lower dissociations constant than for any other molecule present in a sample) binding with high affinity, e.g. with a dissociation constant of 10⁻⁴, more preferably 10⁻⁶, yet more preferably 10⁻⁸ or in each case lower, to the respective molecule of interest.

The determination of the phosphorylation status of an FRS-2 (this term wherever used including FRS-2, a variant thereof (especially as defined above) or a fragment thereof comprising an (unsphosphorylated and/or phosphorlyted) tyrosine) may, for example, include (at least partial) purification of FRS-2 or a variant or a tyrosine comprising fragment thereof from a biological sample, e.g. including lysis of tissues, cells or cell fragments and one or more enriching steps, e.g. by classical chromatographic or Fast Protein Liquid Chromatography (FPLC) and/or electrophoretic techniques, such as two-dimensional electrophoresis or especially sodium dodecylsulfate polyacrylamide gel electrophoresis (SDS-PAGE), more preferably by immunoprecipitation with an FRS-2 specific biospecific recognition reagent, e.g. an antibody or antibodies, more preferably a polyclonal antibody, followed by SDS-PAGE, or by any appropriate combination of two or more such techniques, followed by screening for phosphorylated FRS-2, a variant or fragment thereof for determining the presence or the quantitative amount of phosphorylated (especially tyrosin phosphorylated) FRS-2, variant or fragment or the phosphotyrosine content in the FRS-2 or variant or fragment, with a (itself unlabeled or labeled) biospecific recognition reagent that recognizes the phosphorrylated FRS-2, variant or fragment (especially phosphotyrosine comprised therein).

“Partial purification” means that at least a two-fold, more preferably an at least 5-fold, yet more preferably an at least 10-fold, most preferably an at least 25-fold enrichment of FRS-2, a variant or a fragment thereof is made, compared relatively to other proteins originally present in the sample.

Alternatively, a biospecific recognition reagent recognizing FRS-2 may also be bound to a solid support (such as a membrane or a reaction vessel, e.g. a multi-well plate), e.g. covalently or by adsorption, contacted with a medium comprising the FRS-2 to be assessed (e.g. a tissue or cell lysate) and then the amount of phosphorylated (especially tyrosine-phosphorylated) FRS-2 bound be determined by a biospecific recognition reagent capable of recognizing and labeling phosphorylated FRS-2, especially phosphotyrosine comprised therein.

Yet alternatively, a biospecific recognition reagent specific for a phosphorylated form of FRS-2, especially for phosphotyrosine, may be bound to a solid support (see last paragraph), contacted with a medium comprising the FRS-2 to be assessed and then the FRS-2 bound be determined using a biospecific recognition reagent capable to recognize and label the bound FRS-2.

The labeling step, in each case, preferably comprises one or more further labeled specific recognition molecules that are capable to bind to antibodies bound to their epitope, such as labeled protein A, labeled protein G, labeled streptavidin, labeled further antibodies e.g. specific for the constant region of antibodies or the like.

In principle, also one-step determinations are possible, e.g. with biospecific recognition reagents that are specific for phosphorylated forms of FRS-2, including variants or fragments thereof, and allow to label and/or isolate it .

Further, it is also possible to determine both the phosphorylated (e.g. by biospecific recognition reagents binding only to the phosphorylated forms) and the unphosphorylated forms (e.g. by biospecific recognition reagents binding only the unphosphorylated form) of FRS-2 in a sample (e.g. in order to obtain their ratio) and thus to obtain more detailed information about subtle differences in the samples, including the possibility of quantification.

These or various other methods for determining the phosphorylation status of an FRS-2 are possible and thus part of the invention.

Solid supports for recognition agents or FRS-2, a variant thereof or a tyrosine comprising fragment thereof, or for immunoprecipitates, may, for example, be plastic or glass vessels or plates or multi-well plates customary in cell and immunochemistry, or they may be membranes, e.g. nitrocellulose or PVDF membranes.

In the determining steps, for the labeling the biospecific recognition reagent or a further labeled specific recognition molecule (as defined above and below) binding to it is preferably labeled in a customary way, e.g. by enzyme conjugation e.g. with a peroxidase, such as horseradish peroxidase, thus allowing to determine the presence of bound peroxidase-conjugated recognition agent with customary reactions, such as reaction with dyes or other reagents, e.g. using the SuperSignal®West Dura Extended Duration Substrate detection system (Pierce, Pierce Biotechnology, Inc., Rockford, Ill., USA; #34075, comprising luminal and an enhancer for light intensity, or staining with 4-chloro-1-naphthol and H₂O₂, with alkaline phosphatase (using the phosphatase/BCIP-NBT system), with β-galactosidase; with malate dehydrogenase, with staphylococcal nuclease, with delta-5-steroid isomerase, with yeast alcohol dehydrogenase, with alpha-glycerophosphate dehydrogenase, with triose phosphate isomerase or with glucoamylase, labeling with a component of the biotin/streptavidin system (the one not bound to the biospecific recognition reagent), chromogenic, fluorescent (e.g. fluorescent Europium chelate or Cy5), bioluminescent or chemiluminescent markers, such as fluorescein isothiocyanate, rhodamine, phycoerythrin, phycocyanin, allophycocyanin, o-phthalaldehyde, fluorescamine or fluorescence-emitting metal atoms such as europium or other lanthanides, or radioactive labels, or the like, in each case allowing for known and especially standard detection reactions well known in the art, thus allowing e.g. for enzyme-, color-, chemiluminescence-, fluorescence-, bioluminescence- or radioactivity-based (e.g. fluorescence) or other detection and quantification methods.

Alternatively, instead of directly labeling a biospecific recognition molecule binding to phosphorylated FRS-2, a variant or a tyrosine comprising fragment thereof, further labeled biospecific recognition molecules, such as labeled antibodies or bacterial proteins (e.g. protein A or protein G) (labeled e.g. as just mentioned) may be used that, e.g., bind to constant regions or oligosaccharides on the biospecific recognition molecules, e.g. antibodies, already bound (e.g. labeled anti-mouse or anti-rabbit AB) and thus allow for the determination of bound antibodies can be used, as is known in the art.

Phosphorylated (especially tyrosine-phosphorylated) FRS-2 can be specifically recognized in each case by biospecific recognition agents (especially antibodies) that allow to specifically recognize conformational and/or primary structure based epitopes of phosphorylated FRS-2 or fragments thereof comprising phosphorylated groups, especially by antiphosphotyrosine antibodies.

“Recognizing” or “recognize” preferably means specifically binding to, e.g. with the dissociation constants indicated for biospecific recognition agents.

A variety of antiphosphotyrosine antibodies are, for example, available commercially from a number of sources, are suitable for the method of the present invention. For example, PY-7E1, PY-1B2, and PY20 are monoclonal mouse antiphosphotyrosine antibodies available from Zymed (San Francisco, Calif.) individually or as a cocktail sold under the trademark PY-PLUS.TM. Zymed also offers an affinity-purified polyclonal rabbit antiphosphotyrosine antibody, Z-PY1. A mouse antiphosphotyrosine antibody, clone PT-66 is available from Sigma (St. Louis, Mo.). Furthermore, polyclonal phosphotyrosine antibodies may be raised in a variety of species according to immunization methods well known in the art. A method for the production of monoclonal antiphosphotyrosine antibodies is described in U.S. Pat. No. 4,543,439, the contents of which, especially regarding the methods of obtaining and testing of antiphosphotyrosine antibodies (especially the selection system for specificity which can also be used to screen for other antiphosphotyrosine antibodies) and obtainable antiphosphotyrosine antibodies, are hereby incorporated by reference.

For immunoprecipitation purposes, polyclonal or bispecific antibodies (binding to two different epitopes on the antigen, especially FRS-2) can be used (allowing for the formation of large antibody/antigen agglomerates) are especially preferred, for binding to a solid support, any of the antibodies or derivatives thereof mentioned still showing (especially specific) binding activity are possible.

In one preferred variant, a method or use according to the invention comprises first (at least partially) purifying, e.g. by precipitating or binding, FRS-2, a variant thereof or a (unphosphorylated or phosphorylated) tyrosine comprising fragment thereof (any of these variations referred to as “FRS-2x” hereafter) from a biological sample, e.g. with a first biospecific recognition reagent (e.g. antibody) capable of recognizing FRS-2x and then separating the FRS-2x from the biospecific recognition reagent, e.g. by SDS-PAGE, immobilizing the FRS-2x, e.g. by blotting the FRS-2x to a membrane, then binding a second biospecific recognition reagent (which may itself be labeled, so that no further labeling reaction is required, or unlabeled) capable of recognizing phosphorylated FRS-2x, especially phosphotyrosine, and (if the second biospecific recognition reagent is not already labeled itself) further binding a labeled biospecific recognition reagent (the labels and labeled biospecific recognition reagents may preferably be as defined above) to the second biospecific recognition reagent bound to the FRS-2x and identifying the bound labeled biospecific recognition reagent. Thus, it can be seen whether or to which extent phosphorylated, especially tyrosine-phosphorylated, FRS-2x is present in the sample.

Where “tyrosine comprising fragments” are mentioned throughout this disclosure, they can be in unphosphorylated and/or (preferably) in phosphorylated form.

Immunoprecipitation, labeling, blotting and other methods used herein can be deduced from standard works such as Harlow et al., Antibodies: A Laboratory Manual, Cold Spring Harbor Laboratory, 1988; Coligan et al., Current Protocols in Immunology, Wiley Interscience, 1991; E. Bast: Mikrobiologische Methoden ((Microbiologic Methods)), 2^{nd} edition, Spektrum Akademischer Verlag, Heidelberg/Berlin, 2001; Hans Günter Gassen/Gangolf Schrimpf (eds.), Gentechnische Methoden, 2^{nd} edition Spektrum Akademischer Verlag, Heidelberg/Berlin 1999) , all of which are preferably incorporated by reference herein.

A method or use, in a special embodiment according to the invention, may also comprise comparing the (especially tyrosine) phosphorylation status of FRS-2 or a variant or a tyrosine comprising fragment thereof from a biological patient sample with a previously determined range in samples obtained from patients that show no sensitivity to inhibition of signaling into which an FGF-R is involved and/or from patients that shows such sensitivity in order to determine whether the phosphorylation status is sufficient to deduce a sensitivity towards inhibition of signaling into which a FGF-R is involved. The higher the phosphorylation ratio, the higher the susceptibility to inhibition of FGF-R signaling to be expected.

Dissociation constants, where mentioned, are preferably measured in phosphate buffered saline pH 7.4 which can be prepared as follows: A 10 liter stock of 10× PBS can be prepared by dissolving 800 g NaCl, 20 g KCl, 144 g Na₂HPO₄ and 24 g KH₂PO₄ in 8 L of distilled water, and topping up to 10 L. The pH is -6.8, but when diluted to 1x PBS it should change to 7.4. On dilution, the resultant 1x PBS will have a final concentration : 137 mM NaCl, 10 mM Phosphate, 2.7 mM KCl, pH 7.4.

As understood herein, the term “biomarker” (or “marker”) refers to a biological molecule (here especially unphosphorylated or phosphorylated FRS-2, or a variant or a tyrosine comprising fragment thereof) the presence and/or concentration of which can be detected and correlated with a known condition, especially a disease or disorder state that depends on signaling into which a FGF-R is involved and/or that shows sensitivity. It also includes fragments (obtainable e.g. by protease treatment (e.g. with a site-specific protease) or the like of FRS-2) that still allow to determine the phosphorylation status (especially comprise phosphorylated tyrosine) of FRS-2 or a variant thereof. Such biomarkers are differentially present in subjects suffering from conditions, such as diseases or disorders, responsive to inhibition of signaling into which FGF-R or a variant thereof thereof is involved and patients with diseases that are not responsive to such inhibition.

The invention also relates to a kit that allows to show sensitivity to inhibition of signaling into which an FGF-R or a variant thereof is involved, comprising means for determining the phosphorylation status of an FRS-2, a variant thereof or a tyrosine comprising fragment thereof, especially means for identifying phosphorylated, more especially tyrosine phosphorylated FRS-2, a variant thereof or a tyrosine comprising fragment thereof, in a biological sample as biomarker for such sensitivity to inhibition.

A kit according to the invention can, for example, be a kit for sandwich immunoassay (ELISA) (including sandwich ELISA or competitive ELISA), for fluorescence-based immunoassays or for other immunoassays or enzyme immunoassays, such as Surface-enhanced laser desorption/ionization (SELDI)-based immunoassays, Western blots, immunoprecipitation, immunohistochemistry, immunofluorescence, radioimmunoassay (RIA) and/or immunoradiometric assay (IRMA). The components and ingredients required for such assays are known in the art, and a kit according to the invention comprises at least two such components that allow to identify the phosphorylation status of an FRS-2, a variant thereof or a tyrosine comprising fragment thereof

The kits may comprise biochips, e.g. protein biochips adapted for the capture of protein, e.g. from Ciphergen Biosystems, Inc. (Fremont, Calif., USA), Packard Bioscience Co. (Meriden, Conn., USA), Zyomyx (Hayward, Calif., USA), Phylos (Lexington, Mass., USA) or Biacore (Uppsala, Sweden), which are e.g. described in U.S. Pat. No. 6,225,047; WO 99/51773; U.S. Pat. No. 6,329,209; WO 00/56934; or U.S. Pat. No. 5,242,828.

Preferably, a kit according to the invention comprises—as means for determining the phosphorylation status of an FRS-2, a variant thereof or a tyrosine comprising fragment thereof - a biospecific recognition reagent capable of recognizing, especially binding to, phosphorylated FRS-2, a variant thereof or a fragment thereof comprising (preferably phosphorylated) tyrosine, especially an antibody, more especially an antiphosphotyrosine antibody; and yet more preferably in addition—as a means for at least partial purification of an FRS-2, a variant thereof or a tyrosine comprising fragment thereof—a biospecific recognition reagent capable of recognizing, preferably of immunoprecipitating, FRS-2, a variant thereof or a fragment thereof comprising tyrosine, and labels for identifying said biospecific recognition reagent capable of recognizing, especially binding to, FRS-2, a variant thereof or a fragment thereof comprising (preferably phosphorylated) tyrosine. The labels may preferably be in the form of additional biospecific recognition molecules, e.g. as defined above, that recognize, especially bind to, the biospecific recognition reagent capable of recognizing, especially binding to, phosphorylated FRS-2, a variant thereof or a fragment thereof comprising (phosphorylated) tyrosine, in a state where the latter is bound to phosphorylated FRS-2, a phosphorylated variant thereof or a fragment thereof comprising phosphorylated tyrosine, that are labeled as described above, such as labeled protein A, labeled protein G, labeled streptavidin, labeled further antibodies e.g. specific for the constant region of antibodies or the like.

Compounds allowing for modulation, especially inhibition, of signaling into which a FGF-R or a variant thereof is involved, are especially modulators, preferably inhibitors, which may, for example, be selected from the group consisting of: (especially humanized) antibodies, fragments thereof, single chain antibodies, or especially other chemical agents, especially inhibitors, e.g. one or more of those mentioned in U.S. Pat. No.6,774,237 (which is incorporated by reference herein, especially with regard to the compounds (end products) falling under the claims, more preferably the compounds mentioned therein specifically as examples), 3-(2,6-dichloro-3,5-dimethoxyphenyl)-1-{6-[4-(4-ethyl-piperazin-1-yl)-phenylamino]-pyrimidin-4-yl}-1-methyl urea (also called inhibitor 1 herein, see Example 1), other compounds falling under the claims of, or especially mentioned in, WO 2006/000420 A (which is incorporated by reference herein, especially with regard to the compounds (end products) falling under the claims, more preferably the compounds mentioned therein specifically as examples), PD17307 (inhibitor 2), or other compounds (end products) falling under the claims, or especially mentioned in, U.S. Pat. No. 5,733,913 (which is incorporated by reference herein, especially with regard to the final compounds falling under the claims, more preferably the compounds mentioned therein specifically as examples), PD166866 (J. Med. Chem. 40: 2296-2303, 1997).

Throughout the description and claims of this specification, the words “comprise” and “include” and variations of the words, for example “comprising” and “comprises”, usually mean “including but not limited to”, and are not intended to (and do not) exclude other moieties, additives, components, integers or steps, in contrast to “contain” and variations thereof, such as “contains” or “containing”, which mean that the components or features to which this word is attributed are limited to those mentioned. Where “comprises” or “comprising”is used, where appropriate and reasonable this can be replaced by “consists of” or “consisting of”.

Any mentioning of documents or references in the present disclosure is not intended not mean an admission that the referenced material is prior art negatively affecting the patent-ability and scope of the invention.

### DESCRIPTION OF THE FIGURES

FIG. 1: FRS-2 tyrosine phosphorylation in the cell lines given in the figure. Exponentially growing cells are lysed (see Example 1) and total cell lysates are subjected to immunoprecipitation with an α-FRS-2 antibody followed by immunoblotting with anti-pTyr antibody or -anti-FRS-2 antibody, or directly subjected to immunoblotting with anti-pFRS2 antibody given in Table 1 in Example 1. WB=Western Blot(ting), IP=Immunoprecipitation.

FIG. 2: Comparative analysis of FRS-2 phosphorylation following treatment with the FGF-R inhibitor PD173074 (inhibitor 1, see Methods in Example 1) in the RT4 cells, in the case of RT112 with TKI258/CHIR258, 4-Amino-5-fluoro-3[6-(4-methyl-1-piperazinyl)-1H-benzimidazol-2-yl]-2(1H)-quinolinone (inhibitor 2, see Methods in Example 1). The cell lines indicated are treated as shown. FRS-2 tyrosine phosphorylation is determined by immunoprecipitation with a specific FRS-2 antibody followed by anti-FRS-2 Western Blotting, or by western blot using an antibody that detects phosphorylated Tyr196 on FRS2. Note: FRS-2 protein often shows different electrophoretic mobility shifts caused by phosphorylation on serine/threonine residues by MAPK (Lax et al., Mol. Cell 10: 709-719, 2004). Inhib. 1=inhibitor 1, Inhib. 2=inhibitor 2, DMSO=dimethyl sulfoxide.

(A)=Bladder cancer cell line RT4

(B)=Bladder cancer cell line RT112

FIG. 3: Comparative analysis of FRS-2 tyrosine phosphorylation upon growth factor stimulation: The bladder cancer cell lines RT112 and RT2 are stimulated with aFGF/heparin (50 ng/ml//5 μg/ml), EGF (10 ng/ml) (from R & D Systems, Inc., Minneapolis, Minn., USA; #236-EG-200) or insulin (5 μg/ml) (Sigma, Sigma-Aldrich, Inc., St. Louis, Mo., USA; #1882). Cells are lysed and total protein lysates are recovered. FRS-2 tyrosine phosphorylation is determined by immunoprecipitation followed by anti-pTyr antibody Western Blotting, using antibodies mentioned in Table 1 of Example 1. Total cell lysates are examined for MAPK activation by Western Blotting with anti-phosphoMAPK (pMAPK antibody in Table 1 in Example 1). Equal amount of MAPK protein is monitored by Western Blotting with α-MAPK (MAPK in Table 1 in Example 1). WB=Western Blotting, IP=Immunoprecipitation, Untr.=untreated, INS=insulin.

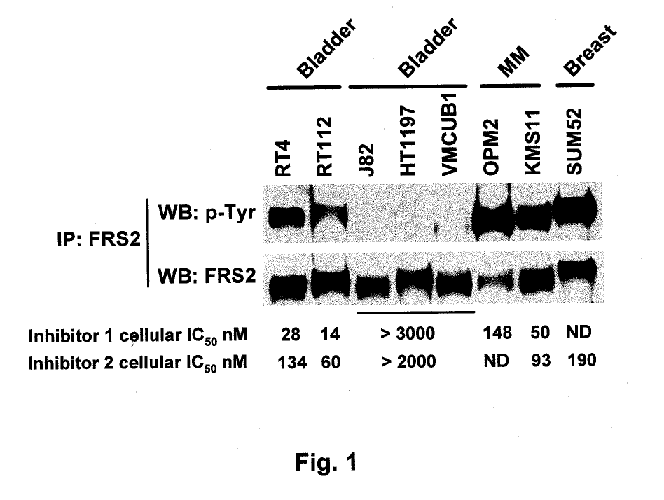

In FIGS. 1 to 3, referring to Table 1 in Example 1, antibody # sc-8318 from Santa Cruz is used for immunoprecipitation when the Western Blot is done with the PTyr antibody, antibody #05-502 is used for immunoprecipitation when the Western Blot is done with the anti-FRS-2 antibody. The antibody to do the Western Blot for FRS-2 is # Sc-8318 from Santa Cruz. “#” stands for catalogue number.

### PREFERRED EMBODIMENTS OF THE INVENTION

In the following, some preferred embodiments of the invention are mentioned—other preferred embodiments can, as mentioned above, be obtained by replacing one or more terms describing the embodiments by definitions given above or in the Examples.

(A1) In one preferred embodiment, the invention relates to a method of identification of cells that show sensitivity to modulation, especially inhibition of signaling into which a Fibroblast Growth Factor Receptor (FGF-R) or a variant thereof is involved, comprising determining the phosphorylation status of an FGF-R substrate 2 (FRS-2), a variant thereof or a tyrosine comprising fragment thereof in a biological sample as biomarker for such sensitivity to inhibition, wherein the phosphorylation status of tyrosine of an FRS-2 is used as the biomarker.

(A2) Another preferred embodiment of the invention relates to the method according (A2), wherein a positive finding of phosphorylation, especially of tyrosine, in FRS-2 or a variant thereof, in the absence of a modulator, especially of FGF, is used as indication that inhibition of signaling into which a Fibroblast Growth Factor Receptor (FGF-R) or a variant thereof is involved can be effective to affect the signaling, especially to inhibit the signaling.

(A3) Another preferred embodiment of the invention relates to the method according to (A2) or (A3), wherein the phosphorylation status of FRS-2, a variant thereof or a tyrosine comprising fragment thereof in the biological sample after incubation in the presence and the absence of an inhibitor of signaling into which a Fibroblast Growth Factor Receptor (FGF-R) or a variant thereof is compared in order to identify cells that are responsive to administration of the inhibitor, where a finding of inhibition of the phosphorylation is taken as indication that such responsiveness is to be expected.

(A4) Another preferred embodiment of the invention relates to the method according to any one of (A1) to (A3), wherein the term FGF-R or variants includes all those forms or variants of FGF-R that still, active due to binding—preferably with a dissociation constant of 10⁻³ or stronger, more preferably of 10⁻⁵ or stronger, yet more preferably of 10⁻⁷ or stronger—of one or more Fibroblast Growth Factor, or preferably constitutionally active, are able to phosphorylate FRS-2 to yield the phosphotyrosine form thereof, as demonstrable with an antiphosphotyrosine antibody, and that comprise, preferably consist of, a sequence that is 70% or more identical, more preferably at least 85% or more identical, yet more preferably 90% or more identical, still more preferred 95% or more identical, very preferred 98% or more identical when compared with one of FGF-R1, FGF-R2, FGF-R3 or FGF-R4, respectively, and wherein the term FGF-R substrate 2 (FRS-2), a variant thereof or a tyrosine comprising fragment thereof includes those forms of FRS-2 which still are able to bind to FGF-R1, FGF-R2, FGF-R3 and/or FGF-R4, especially FRS-2 variants that are 70% or more identical, more preferably at least about 85% or more identical, yet more preferably about 90% or more identical, still more preferred about 95% or more identical, very preferred 98% or more identical, to FRS-2a or FRS-213, or fragments thereof that comprise a phosphotyrosine.

(A5) Another preferred embodiment of the invention relates to the method according to any one of (A1) to (A4), comprising at least partially purifying FRS-2, a variant thereof or a tyrosine comprising fragment thereof and then determining the presence or the amount of phosphorylated, especially tyrosine phosphorylated with a biospecific recognition reagent capable of recognizing a phosphorylated form of FRS-2, of a variant or of a fragment thereof, especially phosphotyrosine comprised therein, wherein either said biospecific recognition reagent or a further biospecific recognition molecule is administered capable of binding to said biospecific recognition reagent is labeled and is administered, thus allowing for detection of the phosphorylated form of FRS-2, of the variant or of the fragment thereof.

(A6) Another preferred embodiment of the invention relates to the method of (A5), wherein the biospecific recognition reagent is an antibody.

(A7) Another preferred embodiment of the invention relates to the method according to any one of (A1) to (A6), wherein phosphotyrosine-comprising FRS-2, a phosphotyrosine comprising variant thereof or a phosphotyrosine comprising fragment thereof is used as biomarker indicative for cells that show sensitivity to modulation, especially inhibition of signaling into which a Fibroblast Growth Factor Receptor (FGF-R) or a variant thereof is involved, and preferably comprising using a biospecific recognition reagent in the form of an antiphosphotyrosine antibody to determine the presence or amount of tyrosine phosphorylation in said FRS-2, variant or fragment thereof.

(A8) Another preferred embodiment of the invention relates to the method according to any one of (A1 to A7) wherein the cells that are sensitive to modulation, especially inhibition, of

FGF-R signaling are distinguished from such cells that proliferate independently of FGF-Rs, especially where the cells sensitive to inhibition show FGF-independent, more especially constitutive, phosphorylation of FRS-2, a variant thereof or a tyrosine comprising fragment thereof, is found, especially tyrosine phosphorylation.

(A9) Another preferred embodiment of the invention relates to the method according to any one of (A1) to (A8), wherein an absence of phosphorylation of FRS-2, especially absence of phosphorylation in the absence of FRS-2, of a variant or of a tyrosine comprising fragment thereof is taken as evidence that inhibition of FGF-R signaling by an inhibitor of FGF-R signaling is not to be expected.

(B1) Alternatively, the invention preferably relates to a method of using or the use of phosphorylation (especially phosphotyrosine) identification in FRS-2, a variant thereof or a tyrosine comprising fragment thereof, as a biomarker for cells, tissues or organs that show hyperactive, especially constitutively activated, FGF-R signaling, especially that are treatable with inhibitors of FGF-R or a variant thereof and that are responsive to such inhibitors, said method or use comprising determining the presence of phosphorylated tyrosine in FRS-2, in a variant thereof or in a tyrosine comprising fragment thereof from a biological sample with a biospecific recognition reagent capable of recognizing phosphotyrosine in FRS-2, a positive finding of phosphorylation indicating hyperactive, especially constitutively activated, FGF-R signaling

(B2) Another preferred embodiment of the invention relates to the method according to (B1), further including, in order to distinguish cells or tissues or organs that are responsive from such cells or tissues or organs that are non-responsive to inhibitors of signaling into which an FGF-R or a variant thereof is involved, comparing the tyrosine phosphorylation status in the absence and in the presence of an inhibitor of signaling mediated by FGF-R or a variant thereof, a decrease in the tyrosine phosphorylation in the presence of an inhibitor indicating such responsiveness.

(B3) Another preferred embodiment of the invention relates to the method according to (B1) or (B2), wherein the tyrosine phosphorylation degree of FRS-2, of a variant thereof or of a tyrosine comprising fragment thereof in the biological sample after incubation in the presence and the absence of an inhibitor of signaling into which a Fibroblast Growth Factor Receptor (FGF-R) or a variant thereof are compared in order to identify cells that are responsive to administration of the inhibitor, where a finding of partial or complete inhibition of the phosphorylation, that is, a decrease in phosphorylation, is taken as indication that such responsiveness is to be expected.

(B4) Another preferred embodiment of the invention relates to the method according to any one of (B1) to (B3), wherein the term FGF-R or variants includes all those forms or variants of FGF-R that still, active due to binding—preferably with a dissociation constant of 10⁻³ or stronger, more preferably of 10⁻⁵ or stronger, yet more preferably of 10⁻⁷ or stronger—of one or more Fibroblast Growth Factor, or preferably constitutionally active, are able to phosphorylate FRS-2 to yield the phosphotyrosine form thereof, as demonstrable with an antiphosphotyrosine antibody, and that comprise, preferably consist of, a sequence that is 70% or more identical, more preferably at least 85% or more identical, yet more preferably 90% or more identical, still more preferred 95% or more identical, very preferred 98% or more identical when compared with one of FGF-R1, FGF-R2, FGF-R3 or FGF-R4, respectively,

and wherein the term FRS-2, a variant thereof or a tyrosine comprising fragment thereof includes those forms of FRS-2 which still are able to bind to FGF-R1, FGF-R2, FGF-R3 and/or FGF-R4, especially FRS-2 variants that are 70% or more identical, more preferably at least about 85% or more identical, yet more preferably about 90% or more identical, still more preferred about 95% or more identical, very preferred 98% or more identical, to FRS-2α or FRS-2β, or fragments thereof that comprise a phosphotyrosine.

(B5) Another preferred embodiment of the invention relates to the method according to any one of (B1) to (B4), comprising at least partially purifying FRS-2, a variant thereof or a tyrosine comprising fragment thereof and then determining the presence or the amount of phosphotyrosine in said FRS-2, in said variant or in said tyrosine comprising fragment thereof using a biospecific recognition reagent capable of recognizing said phosphotyrosine, wherein either said biospecific recognition reagent or a further biospecific recognition molecule capable of binding to said biospecific recognition reagent is labeled and is administered, thus allowing for detection of the phosphorylated form of FRS-2, of the variant or of the fragment thereof.

(B6) Another preferred embodiment of the invention relates to the method of (B6), wherein the biospecific recognition reagent is an antiphosphotyrosine antibody.

(B7) Another preferred embodiment of the invention relates to the method according to any one of (B1) to (B6), wherein phosphotyrosine-comprising FRS-2, a phosphotyrosine comprising variant thereof or a phosphotyrosine comprising fragment thereof is used as biomarker indicative for cells that show sensitivity to inhibition of signaling into which a Fibroblast Growth Factor Receptor (FGF-R) or a variant thereof is involved, and preferably comprising using a biospecific recognition reagent in the form of an antiphosphotyrosine antibody to determine the presence or amount of tyrosine phosphorylation in said FRS-2, variant or fragment thereof.

(B8) Another preferred embodiment of the invention relates to the method according to any one of (B1) to (B7) wherein the cells that are sensitive to modulation, especially inhibition, of FGF-R signaling are distinguished from such cells that proliferate independently of FGF-Rs, especially where FGF-independent, more especially constitutive, phosphorylation of FRS-2, a variant thereof or a tyrosine comprising fragment thereof, is found, especially tyrosine phosphorylation.

(B9) Another preferred embodiment of the invention relates to the method according to any one of (B1) to (B8), wherein an absence of tyrosine phosphorylation of FRS-2, of a variant or of a tyrosine comprising fragment thereof is taken as evidence that inhibition of FGF-R signaling by an inhibitor of FGF-R signaling is not to be expected.

(B10) Another preferred embodiment of the invention relates to the method according to any one of (B1) to (B10), comprising
a) contacting the biological sample with a biospecific recognition reagent capable of recognizing FRS-2 or a variant or a tyrosine comprising fragment thereof and
b) determining the phosphorylation status of the tyrosine with a phosphotyrosine biospecific recognition reagent and
c) correlating the phosphorylation status to the sensitivity to inhibition of signaling into which a Fibroblast Growth Factor Receptor (FGF-R) is involved and/or the condition status and/or treatment efficacy.

(C1) A further preferred embodiment of the invention relates to a kit comprising a biospecific recognition reagent for FGF-R or a variant thereof and a biospecific recognition reagent capable of recognizing a phosphorylated form of FRS-2 or of a variant or of a tyrosine comprising fragment thereof for use in the identification of cells from a biological sample, especially cells or tissues or organs, that are sensitive to modulation, especially inhibition, of signaling into which a Fibroblast Growth Factor Receptor (FGF-R) is involved, said kit comprising means for determining the phosphorylation status of an FRS-2, a variant thereof or a tyrosine comprising fragment thereof in a biological sample as biomarker for such sensitivity to inhibition, comprising as means for determining the phosphorylation status, a biospecific recognition reagent capable of recognizing a phosphorylated form of FRS-2 or of a variant or of a tyrosine comprising fragment thereof (especially an antiphosphotyrosine antibody) for use in the identification of cells from cells or tissues or organs that are sensitive to modulation, especially inhibition, of signaling into which a Fibroblast Growth Factor Receptor (FGF-R) is involved, comprising determining the phosphorylation status of an FRS-2, of a variant thereof or of a tyrosine comprising fragment thereof, especially for allowing to determine hyperactivity of FGF-R signaling, more especially constitutive activation of the FGF-R signaling.

(C2) Another preferred embodiment of the invention relates to the kit according to claim (C1) wherein the biospecific recognition reagent capable of recognizing a phosphorylated form of FRS-2 or of a variant or of a tyrosine comprising fragment thereof is an antiphosphotyrosine antibody and the biospecific recognition reagent for FGF-R or a variant thereof is a monoclonal or polyclonal antibody.

(C3) Another preferred embodiment of the invention relates to the kit according to (C1) or (C2) comprising—as means for determining the phosphorylation status of an FRS-2, a variant thereof or a tyrosine comprising fragment thereof—a biospecific recognition reagent capable of recognizing, especially binding to, phosphorylated FRS-2, a variant thereof or a fragment thereof comprising (preferably phosphorylated) tyrosine, especially an antibody, more especially an antiphosphotyrosine antibody; and in addition—as a means for at least partial purification of an FRS-2, a variant thereof or a tyrosine comprising fragment thereof—a biospecific recognition reagent capable of recognizing, preferably of immunoprecipitating, FRS-2, a variant thereof or a fragment thereof comprising tyrosine, and labels for identifying said biospecific recognition reagent capable of recognizing, especially binding to, FRS-2, a variant thereof or a fragment thereof comprising phosphorylated tyrosine.

(D1) Another preferred embodiment of the invention relates to a biospecific recognition reagent capable of recognizing a phosphorylated form of FRS-2 or of a variant or of a tyrosine comprising fragment thereof for use in the identification of cells (especially from a biological sample, more especially from a patient) that show sensitivity to modulation, especially inhibition, of signaling into which a Fibroblast Growth Factor Receptor (FGF-R) or a variant thereof is involved, especially of cells that show hyperactivity, more especially constitutive activation of FGF-R signaling, where said use preferably comprises determining the phosphorylation status of said FRS-2 or variant or fragment thereof; where a finding of phosphorylation in the absence of modulation preferably means that sensitivity to said inhibition can be expected. More preferably, the biospecific recognition agent is for use in the identification of a condition in a patient that is responsive to the treatment with an inhibitor of FGF-R signaling.

(D2) Another preferred embodiment of the invention relates to the biospecific recognition reagent according to (D1) for use in the identification of cells that show a hyperactivity, especially an FGF-independent activation, more especially a constitutive activation of FGF-R signaling.

(D3) Another preferred embodiment of the invention relates to the biospecific recognition reagent according to any one of (D1) to (D2), which is capable of identifying a tyrosine phosphorylated form of FRS-2, a variant thereof or a tyrosine comprising fragment thereof, more preferably which is an antiphosphotyrosine antibody.

(E1) Yet a further preferred embodiment of the invention relates to the use of a biospecific recognition reagent capable of recognizing phosphorylated FRS-2 or a variant or a tyrosine comprising fragment thereof for the manufacture of a diagnostic for the identification of cells from cells or tissues that are sensitive to modulation of signaling into which a Fibroblast Growth Factor Receptor (FGF-R) or a variant thereof is involved, said identification comprising determining the phosphorylation status of an FGF-R substrate 2 (FRS-2), a variant thereof or a tyrosine comprising fragment thereof, wherein the biospecific recognition reagent is capable of identifying a tyrosine phosphorylated form of FRS-2, a variant thereof or a tyrosine comprising fragment thereof, more preferably an antiphosphotyrosine antibody.

(E2) Another preferred embodiment of the invention relates to the use according to (E1), for identification of cells that show a hyperactivity, especially an FGF-independent activation, more especially a constitutive activation of FGF-R signaling.

(F1) Yet another preferred embodiment of the invention relates to the use of a biospecific recognition reagent capable of recognizing phosphorylated FRS-2, a variant thereof or a fragment thereof to identify cells useful for the identification of compounds that modulate FGF-R signaling, wherein the biospecific recognition reagent is capable of identifying a tyrosine phosphorylated form of FRS-2, a variant thereof or a tyrosine comprising fragment thereof, more preferably is an antiphosphotyrosine antibody.

(F2) Another preferred embodiment of the invention relates to the use according to (F1), comprising comparing the phosphorylation degree of FRS-2, a variant or a tyrosine comprising fragment thereof in the absence and in the presence of a known inhibitor of FGF-R signaling, a decrease of the phosphorylation in the presence of the inhibitor indicating cells useful in identifying other inhibitors.

(G1) In an alternative preferred embodiment, the invention relates to a method for identifying cells that proliferate requiring FGF independent, especially constitutive, FGF receptor activation for proliferation and are responsive to inhibition of FGF-R signaling, comprising
a) subjecting a sample of isolated cells or tissue to a medium in the absence of an FGF-R inhibitor and a parallel sample in the presence of an FGF-R receptor inhibitor in the absence of FGF,
b) at least partially purifying FRS-2, a variant thereof or a tyrosine comprising fragment thereof from said samples;
c) determining the phosphorylation status of FRS-2 in said samples; and
d) comparing the phosphorylation status in the samples treated with that in the samples not treated with the inhibitor,
   a decrease of phosphorylation in the presence of an inhibitor indicating cells that are appropriate for identifying inhibitors useful in the treatment of a condition that includes hyperactivity of FGF-R signaling,
   wherein the determination of the phosphorylation status in step c) takes place by means of a biospecific recognition reagent capable of identifying a tyrosine phosphorylated form of FRS-2, a variant thereof or a tyrosine comprising fragment thereof, more preferably by means of an antiphosphotyrosine antibody.

(H1) Yet a further preferred embodiment of the invention relates to a method of using or a use a biospecific recognition reagent capable of recognizing phosphorylated FRS-2, a variant thereof or a tyrosine comprising fragment thereof, for the identification of potential inhibitors of FGF-R dependent signaling, comprising determining with said reagent the phosphorylation status of FRS-2, a variant thereof or a tyrosine comprising fragment thereof, from a biological sample and, in the case of finding of phosphorylation, comparing the degree of phosphorylation in the presence of a test compound with that in its absence, a decrease in the phosphorylation indicating the usefulness of the test compound as inhibitor of FGF-R dependent signaling, wherein the biospecific recognition reagent is capable of identifying a tyrosine phosphorylated form of FRS-2, a variant thereof or a tyrosine comprising fragment thereof, more preferably is an antiphosphotyrosine antibody.

(H2) Another preferred embodiment of the invention relates to the method or use according to H1, wherein the biological sample shows hyperactivity, especially FGF-R independent activity, more especially constitutive activity of FGF-R signaling.

(I1) A further preferred embodiment of the invention relates to a method of diagnosing a disease responsive to treatment with an inhibitor of FGF-R signaling, comprising identifying a phosphorylated form of FRS-2, of a variant thereof or of a tyrosine comprising fragment thereof in a biological sample from a patient, wherein the identifying preferably takes place with a biospecific recognition reagent capable of recognizing a tyrosine phosphorylated form of FRS-2, of a variant thereof or of a tyrosine comprising fragment thereof, especially an antiphosphotyrosine antibody.

(I2). More preferred is the method according to (11) in the identification of a hyperactivity, especially an FGF-independent activation, more especially a constitutive activation of FGF-R signaling, in a biological sample taken from a patient, which is preferably shown by identifying tyrosine-phosphorylated FRS-2, a variant thereof or a fragment thereof comprising a tyrosine moiety, especially also in the absence of EGF or other activators of FGF-R signaling.

In all of the preceding and following embodiments, a showing of lack of phosphorylation preferably serves to identify cells from biological samples that can be expected not to be responsive to inhibition of FGF-R signaling and thus allow, e.g., to identify patients having conditions due to cells or tissues obtained in such sample and thus patients that are not responsive and thus not amenable to treatment with an inhibitor of FGR-R signaling and thus to avoid unnecessary exposure to treatment schedules including such inhibitors, while on the other hand especially allowing to identify patients on the basis of biological samples taken from them that show, preferably constitutive (meaning e.g. in spite of absence of FGF or other FGF-R signaling activators), (especially tyrosine) phosphorylation of FRS-2, a variant thereof or a fragment thereof comprising a tyrosine moiety, and especially are responsive (a biological sample showing diminished tyrosine phosphorylation in the presence of an inhibitor) to an inhibitor of FGF-R signaling and thus can be considered to be amenable to treatment with an inhibitor of FGF-R .signaling as drug.

Most preferred are all embodiments according to the invention that relate to the positive identification of tyrosine phosphorylation in FRS-2, a variant thereof or a tyrosine comprising fragment thereof, in a biological sample in the absence of a modulator of FGF-R signaling, especially an FGF, as this means that cells show FGF-R signaling activity (e.g. hyperactivity or constitutive activity) which should be accessible to inhibition of FGF-R signaling.

When an inhibitor can be expected to have a beneficial effect on a condition to be treated, a biological sample from the respective patient will show that the patient has a diminished degree of phosphorylation of FRS-2, a variant thereof or a tyrosine-comprising fragment thereof during treatment. This is the preferred situation. However, also vice versa, where an activator has a beneficial effect on a condition to be treated, a biological sample from the respective patient will show that the patient has an increased degree of phopshorylation of FRS-2, a variant thereof or a tyrosine-comprising fragment thereof during treatment.

Highly preferred are the methods and components presented in the examples, as well as the antibodies mentioned therein for use in one of the, inventive methods or uses.

The invention also relates to the contents of the abstract which is therefore incorporated here by reference.

### EXAMPLES

The following examples serve to illustrate the invention without limiting its scope.

### Example 1

### Immunoprecipitation and Western Blot to Distinguish Phosphorylation of FRS-2 in Cell Lines

### 1 Methods

1.1 Cell lines and Cell Culture Conditions

(ATCC: American Type Culture Collection, accessible via LGC Promochem GmbH, Mercatorstr. 51, Wesel, Germany or http://www.Igcpromochem-atcc.com/; DSMZ: Deutsche Sammlung von Mikroorganismen and Zeilkulturen GmbH=German Collection of Microorganisms and Cell Cultures, Braunschweig, Germany).

RT112: human urinary bladder transitional cell carcinoma established from a primary bladder carcinoma, histological grade G2, stage not recorded (Masters et al., Cancer Res. 46: 3630-6, 1986). Cells are obtained from DSMZ ACC #418.

RT4: human urinary bladder transitional cell carcinoma established from a recurrent bladder carcinoma, histological grade G1, stage T2. (Masters et al. 1986, loc. cit.). Cells are obtained from ATCC # HTB-2.

VMCUB-1: human urinary bladder transitional cell carcinoma established from a primary bladder carcinoma. Stage and histological grade are not recorded (Masters et al. 1986, loc. cit.). Cells are obtained from DSMZ ACC #400.

J82: human urinary bladder transitional cell carcinoma established from a primary bladder carcinoma, histological grade G3, stage T3 (Masters et al. 1986, loc. cit.). Cells are obtained from ATCC # HTB-1.

HT1197: human urinary bladder transitional cell carcinoma established from a recurrent bladder carcinoma, histological grade G4, stage T2 (Masters et al. 1986, loc. cit.). Cells are obtained from ATCC # CRL-1473.

The bladder carcinoma cell lines are grown in MEM EBS (Minimum Essential Medium with Earls Basal Salts) (Amimed, Allschwil, Switzerland, #1-31F0-I) supplemented with 1% L-glutamine (Amimed #5-10K00-H), 1% MEM NEAA (MEM Non-essential Amino Acid Solution) (Amimed #5-13K00-H), 1% Na- pyruvate (Amimed #5-60F00-H) and 10% FCS (Gibco, Invitrogen AG, Basel, Schweiz, #10082-147).

SUM52: human breast carcinoma cell line derived from a pleural effusion specimen from a breast cancer patient (Ethier et al., Cancer Res. 56: 899-907, 1996; Forozan et al., Br. J. Cancer 81: 1328-34, 1999). This cell line is provided by Dr. N Hynes, Friedrich Miescher Institute, Basel, Switzerland.

SUM52 cells are grown in HAM'S F12 (Amimed #1-14F01-I) supplemented with 1% L-glutamine (Amimed #5-10K00-H), 2% FCS (Gibco #10082-147), 0.1% BSA (Gibco #15260-037), 10 mM HEPES (Gibco #15630-56), 10 μM T3 (Sigma, Sigma-Aldrich, Inc., St. Louis, Mo., USA, # T6397), 1 μg/ml Hydrocortisone (Sigma # H0888), Insulin-Transferin-Selenium-x supplement (Gibco #51500-056).

OPM2 and KMS11: human multiple myeloma (MM) cell lines derived from end-stage disease patients. OPM2 cells are obtained from DSMZ ACC #50.

KMS11 cells are provide by Dr. T Otsuki, Kawasaki Medical School, Okayama, Japan. Both cell lines are reported to carry the t(4,14) translocation and express a mutated, constitutively activated form of FGF-R3. In particular, OPM2 cells harbour a mutation in the ATP binding pocket in the kinase domain, which results in a change of lysine in position 650 into glutamic acid. KMS11 cells harbor a mutation in the extracellular domain of the receptor changing tyrosine in position 373 into cysteine. Both cell lines are grown in RPMI 1640 (Gibco #21875) supplemented with 1% L-glutamine (Amimed #5-10K00-H) and 20% FCS (Gibco #10082-147).

### 1.2 Antibodies

Antibodies used in this reported are listed in Table 1:

TABLE 1
Antibodies
Primary Antibodies

| Epitope/ | | | Appli- |
|---|---|---|---|
| Antigen | Isotype | Source | cation |
| FRS-2 | Rabbit polyclonal | (H-91) Santa Cruz, | WB/IP |
| | | # sc-8318 | |
| FRS-2 | Mouse monoclonal | Upstate Biotechnology, | WB/IP |
| | | # 05-502 | |
| p-FRS2 | Rabbit polyclonal | Cell Signaling, # # 3864 | WB |
| (Tyr196) | | | |
| pMAPK | Rabbit polyclonal | Cell Signaling, # 9101 | WB |
| MAPK | Rabbit polyclonal | Cell Signaling, # 9102 | WB |
| P-Tyr | Mouse monoclonal | 4G10 Upstate Biotech- | WB |
| | | nology, # 05-777 | |
| Mouse IgG | Sheep polyclonal | Amersham # NA931V | WB |
| | HRP-conjugated | | |
| Rabbit IgG | Donkey polyclonal | Amersham # NA934V | WB |
| | HRP-conjugated | | |
| Beta-Tubulin | Mouse Ascites Fluid | TUB 2.1 Sigma # T4026 | WB |

WB: Western Blot;
IP: Immunoprecipitation;
P-tyr: phosphotyrosine
Santa Cruz: Sant Cruz Biotechnology, Inc.
Upstate Biotechnology: Upstate Biotechnology, Inc., now part of Millipore Corp., Billerica, MA, USA.
Cell Signaling: Cell Signaling Technology, Inc., Boston, MA, USA.
Amersham: Amersham plc, Buckinhamshire, United Kingdom, now part of GE Healthcare.

### 1.3 FGF-R Inhibitory Compounds

PD173074 (also called inhibitor 1 herein), an FGF-R specific inhibitor from Parke Davis (see Mohammadi et al., EMBO J. 17: 5896-5904), of which specificity and potency are confirmed. It has the formula:

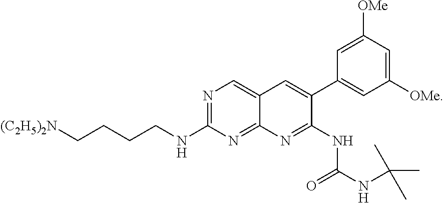

TKI258/CHIR258 (also called inhibitor 2 herein), an FGF-R inhibitor from Chiron of which activity against FGFR1, FGFR2 and FGFR3 are confirmed. It has the formula:

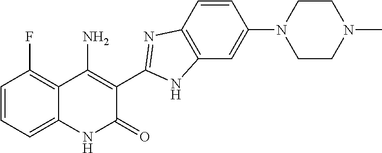

1.4 Immunoprecipitation/WB

Cells are solubilized in 1% Triton extraction buffer containing protease and phosphatase inhibitors (“lysis buffer” 50 mM Tris pH 7.5, 150 mM NaCl, 1 mM EGTA, 5 mM EDTA, 1% Triton, 2 mM NaVanadate, 1 mM PMSF and protease inhibition cocktail from Hoffmann-LaRoche, Basel, Switzerland, #1187358001). Lysates are clarified by centrifugation at 12000×g for 15 min and protein concentration is determined using the DC Protein Assay Reagents (Bio Rad, Bio-Rad Laboratories, Inc., Hercules, Calif., USA, #500-0116) (an assay based on the method of Bradford, M., see Anal. Biochem., 72, 248 (1976), employing Coomassie Blue®, ICI) and a Bovine Serum Albumin (BSA) standard.

Immunoprecipitations are performed by incubating equal amounts of protein with 1 μg of the antibodies indicated in the Figure legends for 2 h on ice. Immunocomplexes are collected with protein A- or protein G-sepharose (Sigma, # P-9424; Sigma, # P-3296) and washed 3 × with lysis buffer. Bound proteins are released by boiling in 2 × sample buffer (20% SDS, 20% glycerol, 160 mM Tris pH 6.8, 4% β-mercaptoethanol, 0.04% bromo-phenol blue).

Samples (total cell lysates or immunocomplexes) are subjected to Sodium Dodecylsulfate Polyacrylaminde Gel Electrophoresis (SDS-PAGE) and proteins blotted onto polyvinyliden fluoride (PVDF) membranes. Prior to adding the primary antibody, filters are blocked in 20% horse serum (inVitromex, Geilenkirchen, Germany; # S0921) or 5% milk in the case of α-FGF-R3 (anti-FGF-R3 antibody, “α-X” generally stands for anti-X-antibody, X standing for the target of the antibody), cyclin D1 or tubulin Western Blots. Proteins are visualized with peroxidase-coupled anti-mouse or anti-rabbit AB using the SuperSignal®West Dura Extended Duration Substrate detection system (Pierce, Pierce Biotechnology, Inc., Rockford, Ill., USA; #34075, comprising luminal and an enhancer for light intensity). Membranes are stripped in 62.5 mM Tris-HCl pH6.8; 2% SDS; 1/125 β-mercaptoethanol for 30 min at 60° C.

### 2 Results

### 2.1 FRS-2 is Tyrosine Phosphorylated in Cancer Cell Lines Dependent on FGF-R Signaling for Proliferation

Since FRS-2 is a substrate for FGF-Rs, we have examined the phosphotyrosine levels of FRS-2 in cell lines that were sensitive to FGF-R inhibitors, and presumably have high FGF-R activity, versus resistant cell lines.

The bladder cancer cell lines RT4 and RTI 12, multiple myeloma lines OPM2 and KMS11, and breast cancer lines SUM52, are dependent on either of the FGF-Rs and their growth is inhibited by inhibitor 1 and/or inhibitor 2. Conversely, the bladder carcinoma lines J82, VMCUB1 or HT1197 are resistant to inhibition by inhibitor 1 and by inhibitor 2. In detail, FGF-R1, FGF-R2 and FGF-R4 expression is determined by Western Blot using specific antibodies, respectively: sc-121 (Santa Cruz), sc-122 (Santa Cruz) and sc-124 (Santa Cruz). To determine the expression of FGF-R3, first FGF-R3 is immunoprecipitated with the antibody F-0425 from Sigma, and the immunocomplexes are subjected to Western Blot using the antibody F-0425 from Sigma. Cell proliferation is measured in 96-well plates. The cells are seeded in a volume of 100 μl per well in the growth media given above. For RT 112, RT4 and SUM52 8500 cells/well are seeded, for VMCUB1, J82, HT1197 and KMS11, 5000 cells/well are seeded, for OPM2 30000 cells/well are seeded. Medium containing FGF-R inhibitor 1, FGFR inhibitor 2 or (as control) DMSO is added 24 h after seeding, respectively. After 72 h, cells are fixed by addition of 25 μl/well glutaraldehyde (20%) for 10 min at room temperature (RT). Cells are then washed twice with 200 μl/well H₂O and 100 μl Methylene Blue (0.05%) are added. After incubation for 10 min at RT, cells are washed 3× with 200 μl/well H₂O. Upon addition of 200 μl/well HCl (3%) and incubation for 30 min at RT on a plate shaker, the Optical Density (OD) at 650 nm is measured. The concentration of inhibitor 1 providing 50% of proliferation inhibition is calculated using Excel module. The results are summarized in Table 2.

The FGF-R dependency as well as sensitivity to inhibitor 1 and/or to inhibitor 2 for each of the indicated cell lines is characterized as indicated above. The effect of inhibitor 1 and inhibitor 2 on cell viability is assessed by means of proliferation assays; the IC50s shown are the average IC50s of several independent assays (their number given by N).

TABLE 2
Summary cancer cell lines

| | | Inhibitor 1 | | Inhibitor 2 | |
|---|---|---|---|---|---|
| Cancer Type | Cell line | IC₅₀ (nM) ± SD | n | IC₅₀ (nM) ± SD | n |
| Bladder | RT112 | 14 ± 3 | 7 | 60 ± 20 | 5 |
| Cancer | RT4 | 28 ± 8 | 15 | 134 ± 58 | 4 |
| | VMCUB1 | >3000 | 2 | >2000 | 3 |
| | J82 | >3000 | 2 | >3000 | 2 |
| | HT1197 | >3000 | 2 | >3000 | 2 |
| Breast | SUM52 | ND | | 190 ± 58 | 4 |
| Cancer | | | | | |
| Multiple | OPM2 | 148 ± 31 | 9 | ND | |
| Myeloma | KMS11 | 50 ± 14 | 4 | 93 | 1 |

SD: Standard Deviation
ND: Not determined
FRS-2 phosphotyrosine levels are elevated in all the cell lines sensitive to FGF-R inhibitors, but undetectable or very low in the resistant cell lines J82, HT1197, VMCUB1 (FIG. 1).

### 2.2 FGF-R Inhibition Blocks FRS-2 Tyrosine Phosphorylation

The specific FGF-R- inhibitor inhibitor 1 and the FGF-R inhibitor inhibitor 2 abolish FRS-2 tyrosine phosphorylation in cancer cell lines shown to be growth inhibited by the compound.

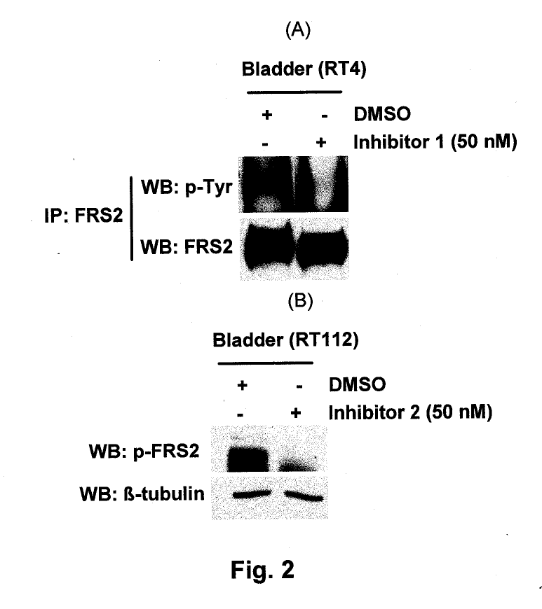

This result indicates that in these cell lines, FGF-Rs are responsible for phosphorylating FRS-2. (FIG. 2).

The indicated cancer cell lines are treated as shown in the legend to FIG. 2 and in FIG. 2. Note: FRS-2 protein often shows differing electrophoretic mobility shifts caused by phosphorylations on serine/threonine residues by MAPK (Lax et al. 2002).

### 2.3 FGF-R but not EGF-R or INS-R Ligand Induces FRS-2 Phosphorylation

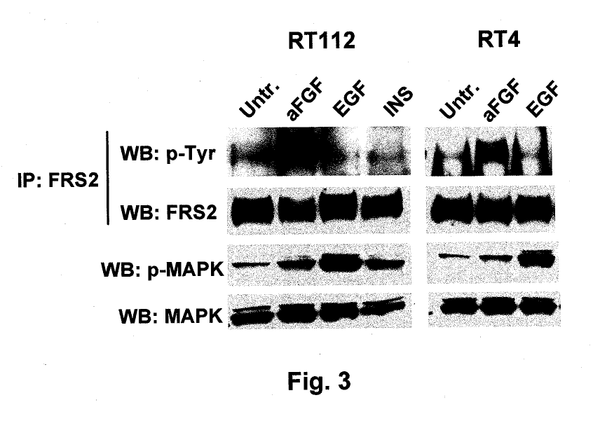

It is examined whether activation of other RTKs not related to the FGF-R can also modulate FRS-2 phosphorylation. EGF and insulin activate MAPK in the RT112 cells, however, they fail to induce phospho-FRS-2. Similarly, EGF strongly induces pMAPK but not phosphoFRS-2 in the RT4 cells. As expected, aFGF efficiently increases FRS-2 phosphotyrosine in the cell lines (FIG. 3).

### 3 Discussion

FRS-2 has been shown to be a downstream substrate of the FGF-R family members and to play a critical role as a docking molecule for multiple proteins involved in FGF-R signal transduction.

We identify a panel of human cancer cell lines that present markedly elevated levels of phospho-FRS-2 and that are very sensitive to FGF-R inhibition, suggesting an active FGF-R-FRS-2 signaling in these lines. In support of this, the specific FGF-R inhibitor inhibitor 1 completely inhibits FRS-2 phosphorylation. In addition, only the FGF-R ligand, aFGF, but not the EGF-R or INS-R ligands, can further increase FRS-2 phosphorylation.

Thus, these results support that phospho-FRS-2 is useful as a biomarker to select cell types, especially patient cell types, and thus patient populations with constitutive FGF-R signaling and therefore, with potential to respond to FGF-R inhibitors.

Further, it is possible to select cell lines for their usefulness for experiments with inhibitors, thus allowing to establish appropriate test systems for the screening of potential drugs.

1. A method of identification of cancer cells that show sensitivity to modulation of signaling into which a Fibroblast Growth Factor Receptor (FGF-R) is involved, comprising determining the phosphorylation status of an FGF-R substrate 2 (FRS-2), in a biological sample as biomarker for such sensitivity wherein reduced level of phosphorylation of FRS-2 indicates that the cancer cells have a reduced likelihood of being sensitive to FGFR inhibition, and increased level of phosphorylation of FRS-2 indicates the cancer cells have an increased likelihood of being sensitive to FGFR inhibition.

2. The method according to claim 1, wherein the phosphorylation status of tyrosine of an FRS-2 is used as the biomarker.

3. The method according to claim 1, wherein a positive finding of phosphorylation in FRS-2, in the absence of a modulator is used as indication that inhibition of signaling into which a Fibroblast Growth Factor Receptor (FGF-R) is involved can be effective to affect the signaling.

4. The method according to claim 3, wherein the phosphorylation status of FRS-2 in the biological sample after incubation in the presence and the absence of an inhibitor of Fibroblast Growth Factor Receptor (FGF-R) signaling pathway is compared in order to identify cells that are responsive to administration of the inhibitor, where a finding of inhibition of the phosphorylation is taken as indication that such responsiveness is to be expected.

5. The method according to claim 1, comprising at least partially purifying FRS-2, and then determining the presence or the amount of phosphorylated, an antibody capable of recognizing a phosphorylated form of FRS-2 comprised therein, wherein either said antibody is labeled and is administered or a further antibody is administered capable of binding to said first antibody is labeled and is administered, thus allowing for detection of the phosphorylated form of FRS-2.

6. The method according claim 1, wherein phosphotyrosine-comprising FRS-2, a phosphotyrosine comprising a phosphotyrosine-comprising fragment thereof is used as biomarker indicative for cells that show sensitivity to modulation of signaling into which a Fibroblast Growth Factor Receptor (FGF-R) is involved, and preferably comprising using an antiphosphotyrosine antibody to determine the presence or amount of tyrosine phosphorylation in said FRS-2, or fragment thereof.

7. The method of claim 3, wherein the phosphorylation is of tyrosine.

8. The method of claim 3, wherein the signaling is inhibited.

9. The method of claim 3 wherein the modulation is inhibition.
